(19) United States
(12) Patent Application Publication
(10) Pub. No.: US 2026/0234178 A1
(43) Pub. Date: Aug. 13, 2026

(54) PYRANOSIDE COMPOUNDS AND METHODS

(71) Applicant: JAMES COOK UNIVERSITY, Townsville (AU)

(72) Inventors: Phurpa WANGCHUK, Townsville (AU); Darren CRAYN, Townsville (AU); Edita RITMEJERYTE, Townsville (AU); Roland RUSCHER, Townsville (AU)

(21) Appl. No.: 19/118,477

(22) PCT Filed: Oct. 5, 2023
(86) PCT No.: PCT/AU2023/050966

§ 371 Date: Apr. 4, 2025

(30) Foreign Application Priority Data
Oct. 5, 2022 (AU) 2022902888

(51) Int. Cl. C07H 15/207; A61K 31/7028; A61K 31/7034; A61P 29/00; C07H 15/18

(52) U.S. Cl. CPC C07H 15/207; A61K 31/7028; A61K 31/7034; A61P 29/00; C07H 15/18

(57) ABSTRACT

The present invention relates to compounds of formula (I) as defined which are biologically active candidates for use in therapy. It also relates to pharmaceutical compositions comprising a compound of formula (I) or formula (II) as defined, methods and uses of compounds of formula (I) and formula (II), and compositions for preventing or treating inflammatory diseases or disorders, especially those associated with an interferon or interleukin, such as inflammatory bowel disease.

### RELATED APPLICATION

This application claims the benefit of Australian Provisional Patent Application No. 2022902888, filed on 5 Oct. 2022, which is incorporated herein by reference in its entirety.

### FIELD OF THE INVENTION

The invention relates to compounds of formula (I) as defined herein which are biologically active candidates for use in therapy. It also relates to pharmaceutical compositions comprising a compound of formula (I) or formula (II) as defined herein, methods and uses of compounds of formula (I) and formula (II), and compositions for preventing or treating inflammatory diseases or disorders, especially those associated with a cytokine, preferably an interferon or interleukin, such as inflammatory bowel disease.

### BACKGROUND OF THE INVENTION

A normal function of the immune system is to detect and destroy or remove foreign materials from the body, which relies on an ability to distinguish between native and foreign materials. An inflammatory disease or disorder is characterised by a dysfunction of the immune system, in which the immune system fails to recognize native materials and attacks normal cells and tissues of the body. This can lead to a variety of symptoms including pain, swelling, fibrosis, damage to cells and tissues, and in severe cases organ failure and even death.

During an immune response, a cause of inflammation includes cytokines, which are released by injured or infected cells. Common cytokines include interleukins and interferons. Interleukins and interferons are signaling molecules. They function to attract immune cells (white blood cells) to the site which elicited the immune response (by chemotaxis) and to up-regulate cellular defense mechanisms. Certain cytokines are known as inflammatory cytokines and are implicated in the inflammatory response and resulting symptoms. These include interferon γ (IFN-γ), interleukin (IL)-17A, and IL-8.

IFN-γ activates macrophages and stimulates neutrophils and natural killer cells. Macrophages and neutrophils are phagocytes which function to destroy and remove foreign materials from the body. Natural killer cells respond to viral infections in particular. One way in which they do this is to release more IFN-γ. (IL)-17A is involved in chemotaxis, being to attract immune cells, including neutrophils, to the site which elicited the immune response. IL-17 also contributes to the release of pro-inflammatory cytokines including IL-6, which in turn stimulates neutrophil production. IL-8 promotes chemotaxis and phagocytosis by neutrophils.

Thus, essentially, an immune response triggers a cascade of events, many of which are upregulated by cytokines, including the release or further cytokines. Moderation of the immune response is often achieved by ejection or destruction of the foreign body and the immune cascade subsides. However, when the immune system fails to recognize its own body as native and attacks it, the cascade of the immune response does not as readily subside. This is a characteristic of an immune disease or disorder. This leads to the symptoms as outlined above which are descriptive of many different diseases and disorders.

One way of treating inflammatory conditions is to artificially moderate the immune response cascade. Suppressing the release of interleukins and interferons is one way of potentially achieving this. There are existing drugs that seek to do this; to suppress the immune response. The most common are glucocorticoids. Glucocorticoids are often effective but unfortunately are associated with severe and potentially dangerous side effects.

One particular inflammatory disease or disorder is Inflammatory Bowel Disease (IDB). IBD encompasses a number of inflammatory conditions of the gastrointestinal tract (GIT) including Crohn's disease and ulcerative colitis (UC) which primarily affect the ileum/colon and colon/rectum, respectively. Crohn's disease and UC are lifelong conditions which damage the digestive tract causing symptoms of varying severity, including rectal bleeding, chronic diarrhoea, pain, vomiting, malabsorption and, in some cases, cancer. Both can also lead to the associated ailments of arthritis, pyoderma gangrenosum and non-thyroidal illness syndrome. While rarely fatal, IBD significantly diminishes quality of life and affects a significant population; approximately 6.8 million people, and presents a substantial social, economic, and healthcare burden.

At a cellular level, IBD is marked by chronic intestinal inflammation caused by dysregulated immune activation, and is one example of an inflammatory condition in which excessive production of proinflammatory cytokines, such as interferon (IFN)-γ. IL-17A and IL-8 lead to the destruction of native gastrointestinal tissue.

Existing management strategies for IBD and other inflammatory conditions are expensive, not effective for all patients, and are often associated with adverse side effects, such as the increased risk of opportunistic infections and malignancies.

Consequently, it would be an advantage to provide alternative treatment options for inflammatory diseases or disorders, including for IBD.

Bio-discovery is a growing field, which investigates and screens for bioactive natural products from natural environments, including plants, microorganisms, coral and other marine life. In the search for bioactive natural products, biological material is screened for molecules having properties that may be of use, for example as therapeutics, pesticides and herbicides. Bio-discovery is one avenue in which alternative therapies are often sought.

### SUMMARY OF THE INVENTION

The present inventors have identified compounds that act to suppress release of interleukins and interferons, and which thus find application in the therapy of associated conditions including inflammatory conditions such as IBD.

In one aspect, the present invention provides a compound of formula (I):

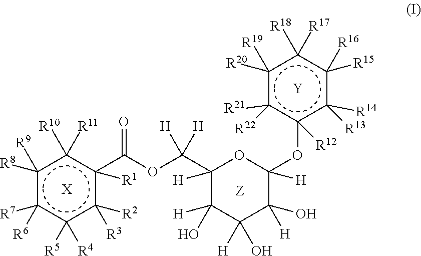

or a pharmaceutically acceptable salt thereof, wherein:
   X is cyclohexane, cyclohexene, cyclohexadiene, or benzene, wherein;
      when X is benzene R¹, R³, R⁵, R⁷, R⁹ and R¹⁰ are absent;
      when X is cyclohexene, either: R¹ and R³ are absent and R¹C—CR²R³ is taken to be C═CR²; R³ and R⁵ are absent and R²R³C—CR⁴R⁵ is taken to be R²C═CR⁴; R⁵ and R⁷ are absent and R⁴R⁵C—CR⁶R⁷ is taken to be R⁴C═CR⁶; R⁷ and R⁹ are absent and R⁶R⁷C—CR⁸R⁹ is taken to be R⁶C═CR⁸; R⁹ and R¹¹ are absent and R⁸R⁹C—CR¹⁰R¹¹ is taken to be R⁸C═CR¹⁰; or R¹¹ and R¹ are absent and R¹⁰R¹¹C—CR¹ is taken to be R¹⁰C═C;
      when X is cyclohexadiene, either: R¹, R³, R⁵ and R⁷ are absent and R¹C—CR²R³ and R⁴R⁵C—CR⁶R⁷ are taken to be C═CR² and R⁴C═CR⁶; R¹, R³, R⁷ and R⁹ are absent and R¹C—CR²R³ and R⁶R⁷C—CR⁸R⁹ are taken to be C═CR² and R⁶C═CR⁸; R¹, R³, R⁹ and R¹¹ are absent and R¹C—CR²R³ and R⁸R⁹C—CR¹⁰R¹¹ are taken to be C═CR² and R²C═CR¹⁰; R³, R⁵, R⁷ and R⁹ are absent and R²R³C—CR⁴R⁵ and R⁶R⁷C—CR⁸R⁹ are taken to be R²C═CR⁴ and R⁶C═CR⁸; R³, R⁵, R⁹ and R¹¹ are absent and R²R³C—CR⁴R⁵ and R⁸R⁹C—CR¹⁰R¹¹ are taken to be R²C═CR⁴ and R⁸C═CR¹⁰; R³, R⁵, R¹¹ and R¹ are absent and R²R³C—CR⁴R⁵ and R¹⁰R¹¹C—CR¹ are taken to be R²C═CR⁴ and R¹⁰C═C; R⁵, R⁷, R⁹ and R¹¹ are absent and R⁴R⁵C—CR⁶R⁷ and R⁸R⁹C—CR¹⁰R¹¹ are taken to be R⁴C═CR⁶ and R⁸C═CR¹⁰; R⁵, R⁷, R¹¹ and R¹ are absent and R⁴R⁵C—CR⁶R⁷ and R¹⁰R¹¹C—CR¹ are taken to be R⁴C═CR⁶ and R¹⁰C═C; or R⁷, R⁹, R¹¹ and R¹ are absent and R⁶R⁷C—CR⁸R⁹ and R¹⁰R¹¹C—CR¹ are taken to be R⁶C═CR⁸ and R¹⁰C═C;
   Y is cyclohexane, cyclohexene, cyclohexadiene, or benzene, wherein;
      when Y is benzene R¹², R¹⁴, R¹⁶, R¹⁸, R²⁰ and R²² are absent;
   when Y is cyclohexene, either: R¹² and R¹⁴ are absent and R¹²C—CR¹³R¹⁴ is taken to be C═CR¹³; R¹⁴ and R¹⁶ are absent and R¹³R¹⁴C—CR¹⁵R¹⁶ is taken to be R¹³C═CR¹⁵; R¹⁶ and R¹⁸ are absent and R¹⁵R¹⁶C—CR¹⁷R¹⁸ is taken to be R¹⁵C═CR¹⁷; R¹⁸ and R²⁰ are absent and R¹⁷R¹⁸C—CR¹⁹R²⁰ is taken to be R¹⁷C═CR¹⁹; R²⁰ and R²² are absent and R¹⁹R²⁰C—CR²¹R²² is taken to be R¹⁹C═CR²¹; or R²² and R¹² are absent and R²¹R²²C—CR¹² is taken to be R²¹C═C;
   when Y is cyclohexadiene, either. R¹², R¹⁴, R¹⁶ and R¹⁸ are absent and R¹²C—CR¹³R¹⁴ and R¹⁵R¹⁶C—CR¹⁷R¹⁸ are taken to be C═CR¹³ and R¹⁵C═CR¹⁷; R¹², R¹⁴, R¹⁸ and R²⁰ are absent and R¹²C—CR¹³R¹⁴ and R¹⁷R¹⁸C—CR¹⁹R²⁰ are taken to be C═CR¹³ and R¹⁷C═CR¹⁹; R¹², R¹⁴, R²⁰ and R²² are absent and R¹²C—CR¹³R¹⁴ and R¹⁹R²⁰C—CR²¹R²² are taken to be C═CR¹³ and R¹⁹C═CR²¹; R¹⁴, R¹⁶, R¹⁸ and R²⁰ are absent and R¹³R¹⁴C—CR¹⁵R¹⁶ and R¹⁷R¹⁸C—CR¹⁹R²⁰ are taken to be R¹³C═CR¹⁵ and R¹⁷C═CR¹⁹; R¹⁴, R¹⁶, R²⁰ and R²² are absent and R¹³R¹⁴C—CR¹⁵R¹⁶ and R¹⁹R²⁰C—CR²¹R²² are taken to be R¹³C═CR¹⁵ and R¹⁹C═CR²¹; R¹⁴, R¹⁶, R²² and R¹² are absent and R¹³R¹⁴C—CR¹⁵R¹⁶ and R²¹R²²C—CR¹² are taken to be R¹³C═CR¹⁵ and R²¹C═C; R¹⁶, R¹⁸, R²⁰ and R²² are absent and R¹⁵R¹⁶C—CR¹⁷R¹⁸ and R¹⁹R²⁰C—CR²¹R²² are taken to be R¹⁵C═CR¹⁷ and R¹⁹C═CR²¹; R¹⁶, R¹⁸, R²² and R¹² are absent and R¹⁵R¹⁶C—CR¹⁷R¹⁸ and R²¹R²²C—CR¹² are taken to be R¹⁵C═CR¹⁷ and R²¹C═C; or R¹⁸, R²⁰, R²² and R¹² are absent and R¹⁷R¹⁸C—CR¹⁹R²⁰ and R²¹R²²═C—CR¹² are taken to be R¹⁷C═CR¹⁹ and R²¹C═C;
   at least one of R¹² to R²² is —C₁-C₁₀ alkyl;
   each of R¹ to R²² when present is otherwise independently selected from the group consisting of —H, —OH, halogen, —CN, —SH, —C₁-C₁₀ alkyl, —C₂-C₁₀ alkenyl, —C₂-C₁₀ alkynyl, —C(O)C₁-C₁₀ alkyl, —C(O)C₂-C₁₀ alkenyl, —C(O)C₂-C₁₀ alkynyl, —OC₁-C₁₀ alkyl, —OC₂-C₁₀ alkenyl, —OC₂-C₁₀ alkynyl, —OC(O)C₁-C₁₀ alkyl, —OC(O)C₂-C₁₀ alkenyl, —OC(O)C₂-C₁₀ alkynyl, —OC(O)NHC₁-C₁₀ alkyl, —OC(O)NHC₂-C₁₀ alkenyl, —OC(O)NHC₂-C₁₀ alkynyl, and —N(R²³)₂, or one or more of R¹³ and R¹⁴, R¹⁵ and R¹⁶, R¹⁷ and R¹⁸, R¹⁹ and R²⁰, and R²¹ and R²², taken together, are ═O, ═S, or ═NR²³ except when Y is cyclohexane in which case not both of R¹⁵ and R¹⁶, and R¹⁹ and R²⁰, taken together, are ═O; and
   each R²³ is independently selected from the group consisting of H and —C₁-C₁₀ alkyl.

In preferred embodiments, Z is α- or β-D-glucose, most preferably β-D-glucose.

In preferred embodiments, X is benzene. Preferably each of R², R⁴, R⁶, R⁸ and R¹⁰ are independently selected from the group consisting of —H and —OH, preferably wherein each of R² and R¹⁰ are —H and each of R⁴, R⁶ and R⁸ are —OH.

In preferred embodiments, Y is cyclohexane, cyclohexene, or benzene. Preferably each of R¹², R¹⁵, R¹⁶, R¹⁹ and R²⁰ when present is independently selected from the group consisting of —H, —OH, —C₁-C₁₀ alkyl and —C(O)C₁-C₁₀ alkyl, preferably —H and —OH. Preferably, each of R¹³, R¹⁴, R¹⁷, R¹⁸, R²¹ and R²² when present is independently selected from the group consisting of —H, —OH, —C₁-C₁₀ alkyl and —C(O)C₁-C₁₀ alkyl, preferably —H, —C₁-C₁₀ alkyl and —C(O)C₁-C₁₀ alkyl, or R¹⁷ and R¹⁸ taken together are ═O, at least one of R¹³, R¹⁴, R¹⁷, R¹⁸, R²¹ and R²² being —C₁-C₁₀ alkyl. Preferably, R¹² is absent or selected from —H and —OH. Preferably, at least one of R¹⁷ and R₁₈ is —C₁-C₁₀ alkyl and the other is absent or —H, or R¹⁷ and R¹⁸ taken together are ═O in which case at least one of R¹³, R¹⁴, R²¹ and R²² is —C₁-C₁₀ alkyl.

Preferably, X is benzene in which case R¹, R³, R⁵, R⁷, R⁹ and R¹¹ are absent, and Y is cyclohexane, cyclohexene, or benzene.

Preferably, each of R¹ to R¹¹ when present is independently selected from the group consisting of —H, —OH, —C₁-C₁₀ alkyl and —C(O)C₁-C₁₀ alkyl, preferably —H and —OH, and each of R¹² to R²² when present is otherwise independently selected from the group consisting of —H, —OH, —C₁-C₁₀ alkyl and —C(O)C₁-C₁₀ alkyl, or one or more of R¹³ and R¹⁴, R¹⁵ and R¹⁶, R¹⁷ and R¹⁸, R¹⁹ and R²⁰, and R²¹ and R²², taken together, are ═O, subject to the proviso that when Y is cyclohexane not both of R¹⁵ and R¹⁶, and R¹⁹ and R²⁰, taken together, are ═O. When ═O is present, preferably one of R¹³ and R¹⁴, R¹⁵ and R¹⁶, R¹⁷ and R¹⁸, R¹⁹ and R²⁰, and R²¹ and R²², taken together, are ═O, preferably wherein R¹⁷ and R¹⁸ taken together are ═O.

Preferably, three of R³, R⁵, R⁷, R⁹ and R¹¹ are —OH, and each of the remainder is —H, each of R¹², R¹⁵, R¹⁶, R¹⁹ and R²⁰ when present is independently selected from the group consisting of —H, —OH, —C₁-C₁₀ alkyl and —C(O)C₁-C₁₀ alkyl, preferably —H and —OH, and the remainder of R¹² to R²² is absent or independently selected from the group consisting of —H, —OH, —C₁-C₁₀ alkyl and —C(O)C₁-C₁₀ alkyl, preferably —H, —C₁-C₁₀ alkyl and —C(O)C₁-C₁₀ alkyl, or R¹ and R¹⁸ taken together are ═O.

Preferably, each of R⁴, R⁶ and R⁸ is —OH, and each of R² and R¹⁰ is —H, R¹² is absent or selected from —H and —OH, at least one of R¹⁷ and R¹⁸ is —C₁-C₁₀ alkyl and the other is absent or —H, or R¹⁷ and R¹⁸ taken together are ═O, and the remainder of R¹² to R²² is absent or independently selected from the group consisting of —H, —OH, —C₁-C₁₀ alkyl and —C(O)C₁-C₁₀ alkyl.

In most preferred embodiments, the compound of Formula (I) is selected from the group consisting of:

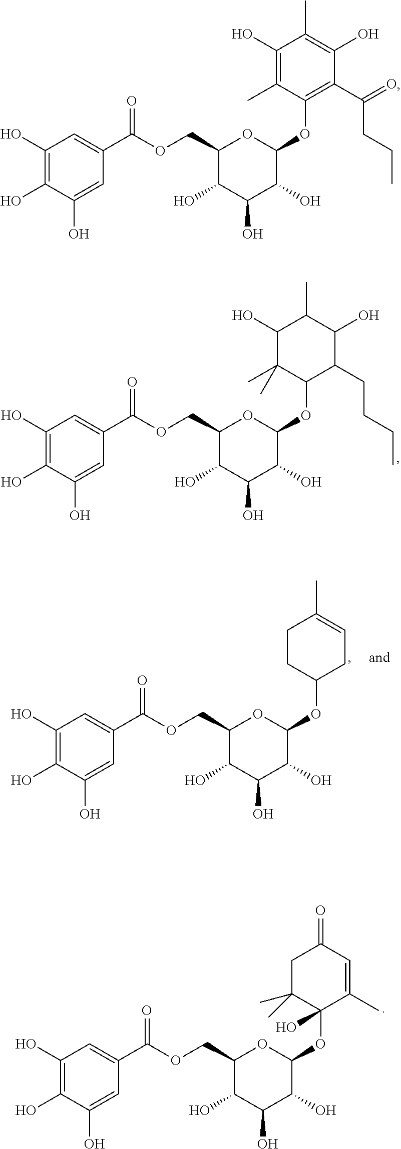 [SMILES: CCCC(=O)c1c(O)c(C)c(O)c(C)c1O[C@@H]1O[C@H](COC(=O)c2cc(O)c(O)c(O)c2)[C@@H](O)[C@H](O)[C@H]1O ; CCCCC1C(O)C(C)C(O)C(C)(C)C1O[C@@H]1O[C@H](COC(=O)c2cc(O)c(O)c(O)c2)[C@@H](O)[C@H](O)[C@H]1O ; CC1=CCC(O[C@@H]2O[C@H](COC(=O)c3cc(O)c(O)c(O)c3)[C@@H](O)[C@H](O)[C@H]2O)CC1 ; CC1=CC(=O)CC(C)(C)[C@@]1(O)O[C@@H]1O[C@H](COC(=O)c2cc(O)c(O)c(O)c2)[C@@H](O)[C@H](O)[C@H]1O]

In another aspect, the present invention provides a method for preventing or treating an inflammatory disease or disorder in a subject, comprising administering to the subject an effective amount of a compound of formula (II):

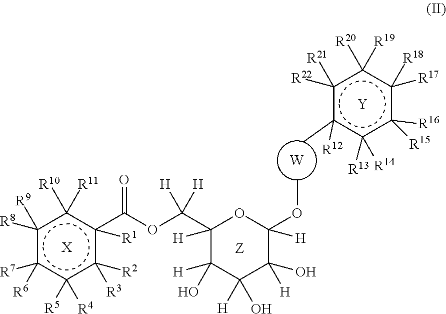

or a pharmaceutically acceptable salt thereof, wherein:
   X is cyclohexane, cyclohexene, cyclohexadiene, or benzene, wherein;
      when X is benzene R¹, R³, R⁵, R⁷, R⁹ and R¹¹ are absent;
      when X is cyclohexene, either: R¹ and R³ are absent and R¹C—CR²R³ is taken to be C═CR²; R³ and R⁵ are absent and R²R³C—CR⁴R⁵ is taken to be R²C═CR⁴; R⁵ and R⁷ are absent and R⁴R⁵C—CR⁶R⁷ is taken to be R⁴C═CR⁶; R⁷ and R⁹ are absent and R⁶R⁷C—CR⁸R⁹ is taken to be R⁶C═CR⁸; R⁹ and R¹¹ are absent and R⁸R⁹C—CR¹⁰R¹¹ is taken to be R⁸C═CR¹⁰; or R¹¹ and R¹ are absent and R¹⁰R¹¹—CR¹ is taken to be R¹⁰C═C;
      when X is cyclohexadiene, either: R¹, R³, R⁵ and R⁷ are absent and R¹C—CR²R³ and R⁴R⁵C—CR⁶R⁷ are taken to be C═CR² and R⁴C═CR⁶; R¹, R³, R⁷ and R⁹ are absent and R¹C—CR²R³ and R⁶R⁷C—CR⁸R⁹ are taken to be C═CR² and R⁶C═CR⁸; R¹, R³, R⁹ and R¹¹ are absent and R¹C—CR²R³ and R⁸R⁹C—CR¹⁰R¹¹ are taken to be C═CR² and R⁸C═CR¹⁰; R³, R⁵, R⁷ and R⁹ are absent and R²R³C—CR⁴R⁵ and R⁶R⁷C—CR⁸R⁹ are taken to be R²C═CR⁴ and R⁶C═CR⁸; R³, R⁵, R⁹ and R¹¹ are absent and R²R³C—CR⁴R⁵ and R⁸R⁹C—CR¹⁰R¹¹ are taken to be R²C═CR and R^{B}C═CR¹⁰; R³, R⁵, R¹¹ and R¹ are absent and R²R³C—CR⁴R⁵ and R¹⁰R¹¹C—CR¹ are taken to be R²C═CR and R¹⁰C═C; R⁵, R⁷, R⁹ and R¹¹ are absent and R⁴R⁵C—CR⁶R⁷ and R⁸R⁹C—CR¹⁰R¹¹ are taken to be R⁴C═CR⁶ and R⁸C═CR¹⁰; R⁵, R⁷, R¹¹ and R¹ are absent and R⁴R⁵C—CR⁶R⁸ and R¹⁰R¹¹C—CR¹ are taken to be R⁴C═CR⁶ and R¹⁰C═C; or R⁷, R⁹, R¹¹ and R¹ are absent and R⁶R⁷C—CR⁸R⁹ and R¹⁰R¹¹C—CR¹ are taken to be R⁶C═CR⁸ and R¹⁰C═C;
   Y is cyclohexane, cyclohexene, cyclohexadiene, or benzene, wherein;
      when Y is benzene R¹², R¹⁴, R¹⁶, R¹⁸, R²⁰ and R²² are absent;
      when Y is cyclohexene, either: R¹² and R¹⁴ are absent and R¹²C—CR¹³R¹⁴ is taken to be C═CR¹³; R¹⁴ and R¹⁶ are absent and R¹³R¹⁴C—CR¹⁵R¹⁶ is taken to be R¹³═CR¹⁵; R¹⁶ and R¹⁸ are absent and R¹⁵R¹⁶C—CR¹⁷R¹⁸ is taken to be R¹⁵C═CR¹⁷; R¹⁸ and R²⁰ are absent and R¹⁷R¹⁸C—CR¹⁹R²⁰ is taken to be R¹⁷C═CR¹⁹; R²⁰ and R²² are absent and R¹⁹R²⁰C—CR²¹R²² is taken to be R¹⁹C═CR²¹; or R²² and R¹² are absent and R²¹R²C—CR¹² is taken to be R²¹C═C;
      when Y is cyclohexadiene, either: R¹², R¹⁴, R¹⁶ and R¹⁸ are absent and R¹²C—CR¹³R¹⁴ and R¹⁵R¹⁶C—CR¹⁷R¹⁸ are taken to be C═CR¹³ and R¹⁵C═CR¹⁷; R¹², R¹⁴, R¹⁸ and R²⁰ are absent and R¹²C—CR¹³R¹⁴ and R¹⁷R¹⁸C—CR¹⁹R²⁰ are taken to be C═CR¹³ and R¹⁷C═CR¹⁹; R¹², R¹⁴, R²⁰ and R²² are absent and R¹²C—CR¹³R¹⁴ and R¹⁹R²⁰C—CR²¹R²² are taken to be C═CR¹³ and R¹⁹C═CR²¹; R¹⁴, R¹⁶, R¹⁸ and R²⁰ are absent and R¹³R¹⁴C—CR¹⁵R¹⁶ and R¹⁷R¹⁸C—CR¹⁹R²⁰ are taken to be R¹³C═CR¹⁵ and R¹⁷C═CR¹⁹; R¹⁴, R¹⁶, R²⁰ and R²² are absent and R¹³R¹⁴C—CR¹⁵R¹⁶ and R¹⁹R²⁰C—CR²¹R²² are taken to be R¹³C═CR¹⁵ and R¹⁹C═CR²¹; R¹⁴, R¹⁶, R²² and R¹² are absent and R¹³R¹⁴C—CR¹⁵R¹⁶ and R²¹R²²C—CR¹² are taken to be R¹³C═CR¹⁵ and R²¹C═C; R¹⁶, R¹⁸, R²⁰ and R²² are absent and R¹⁵R¹⁶C—CR¹⁷R¹⁸ and R¹⁹R²⁰C—CR²¹R²² are taken to be R¹³C═CR¹⁷ and R¹⁹C═CR²¹; R¹⁶, R¹⁸, R²² and R¹² are absent and R¹⁵R¹⁶C—CR¹⁷R¹⁸ and R²¹R²²C—CR¹² are taken to be R¹⁵C═CR¹⁷ and R²¹C═C; or R¹⁸, R²⁰, R²² and R¹² are absent and R¹⁷R¹⁸C—CR¹⁹R²⁰ and R²¹R²²C—CR¹² are taken to be R¹⁷C═CR¹⁹ and R²¹C═C;
   each of R¹ to R² when present is independently selected from the group consisting of —H, —OH, halogen, —CN, —SH, —C₁-C₁₀ alkyl, —C₂-C₁₀ alkenyl, —C₂-C₁₀ alkynyl, —C(O)C₁-C₁₀ alkyl, —C(O)C₂-C₁₀ alkenyl, —C(O)C₂-C₁₀ alkynyl, —OC₁-C₁₀ alkyl, —OC₂-C₁₀ alkenyl, —OC₂-C₁₀ alkynyl, —OC(O)C₁-C₁₀ alkyl, —OC(O)C₂-C₁₀ alkenyl, —OC(O)C₂-C₁₀ alkynyl, —OC(O)NHC₁-C₁₀ alkyl, —OC(O)NHC₂-C₁₀ alkenyl, —OC(O)NHC₂-C₁₀ alkynyl, and —N(R²³)₂, or one or more of R¹³ and R¹⁴, R¹⁵ and R¹⁶, R¹⁷ and R¹⁸, R¹⁹ and R²⁰, and R²¹ and R²², taken together, are ═O, ═S, or ═NR²;
   each R²³ is independently selected from the group consisting of H and —C₁-C₁₀ alkyl; and
   W is absent or selected from the group consisting of C₁-C₁₀ alkylene, C₂-C₁₀ alkenylene, C₂-C₁₀ alkynylene, C(O)C₁-C₁₀ alkylene, C(O)C₂-C₁₀ alkenylene, and C(O)C₂-C₁₀ alkynylene.

Preferred embodiments include those as itemised above in respect of a compound of Formula (I). In these above embodiments, W is absent. In other preferred embodiments. W is selected from the group consisting of C₁-C₁₀ alkylene and C₂-C₁₀ alkenylene.

In most preferred embodiments, the compound of Formula (II) is selected from the group consisting of:

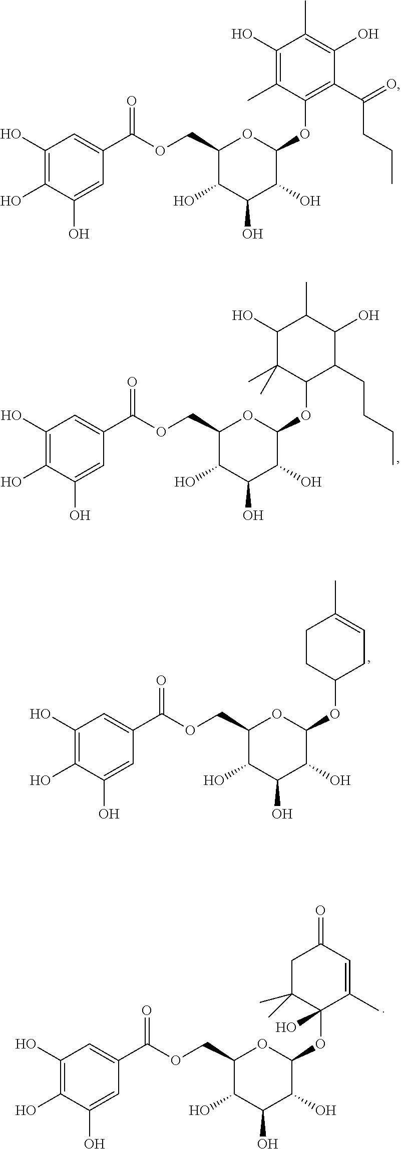 [SMILES: CCCC(=O)c1c(O)c(C)c(O)c(C)c1O[C@@H]1O[C@H](COC(=O)c2cc(O)c(O)c(O)c2)[C@@H](O)[C@H](O)[C@H]1O ; CCCCC1C(O)C(C)C(O)C(C)(C)C1O[C@@H]1O[C@H](COC(=O)c2cc(O)c(O)c(O)c2)[C@@H](O)[C@H](O)[C@H]1O ; CC1=CCC(O[C@@H]2O[C@H](COC(=O)c3cc(O)c(O)c(O)c3)[C@@H](O)[C@H](O)[C@H]2O)CC1 ; CC1=CC(=O)CC(C)(C)[C@@]1(O)O[C@@H]1O[C@H](COC(=O)c2cc(O)c(O)c(O)c2)[C@@H](O)[C@H](O)[C@H]1O]

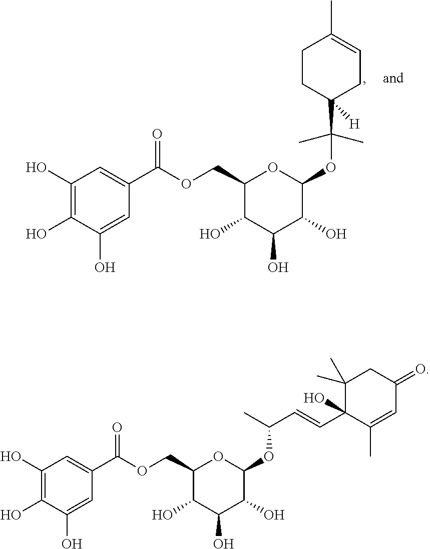

In preferred embodiments, the inflammatory disease or disorder is associated with a cytokine, preferably associated with overexpression of a cytokine, preferably where the cytokine is an interleukin or an interferon. Preferably, the interleukin or an interferon is selected from one or more of the group consisting of IFN-γ, IL-17A and IL-8.

In another aspect, the present invention provides a pharmaceutical composition comprising a compound of Formula (I) or Formula (II) or pharmaceutically acceptable salt as described herein, and one or more pharmaceutically acceptable excipients.

In another aspect, the present invention provides the use of a compound of Formula (I) or Formula (II) or pharmaceutically acceptable salt as described herein, or a pharmaceutical composition as described herein, in the manufacture of a medicament for preventing or treating an inflammatory disease or disorder.

In another aspect, the present invention provides a compound of Formula (I) or Formula (II) or pharmaceutically acceptable salt as described herein, or a pharmaceutical composition as described herein, for use in therapy. Preferably, the therapeutic use is in treating or preventing an inflammatory disease or disorder.

### DESCRIPTION OF THE DRAWINGS

FIG. 1. Analytical high-performance liquid chromatograms (HPLC) of (A) the polar crude extract from Uromytus metrosideros and (B) purity of compounds 1-6 isolated from the polar crude extract from U. metrosideros, both acquired at 254 nm.

FIG. 2. Structures of compounds 1-6 isolated from U. metrosideros.

FIG. 3. HMBC correlations for compounds 1-6 isolated from U. metrosideros.

FIG. 4. Relative stereochemistry determined by NOESY correlations for compound 4 (A) and ROESY correlations for compound 6 (B).

FIG. 5. Cell viability of polar crude extract (PCE) and compounds 3-6 purified from U. metrosideros on PBMC over 24 hours. Cell viability was measured as luminescence (RLU) at (A) 1, (B) 6 and (C) 24 hours, where reduced luminescence indicates cell death. Bar plots show three donor means (±SE), each point represents the donor mean from six technical replicates. Statistical significance was calculated using student's T-test by comparing untreated group to each treatment or lysis buffer control at each time point. **=p<0.01; *=p<0.05; no value=p>0.05.

FIG. 6. The effects of crude extract (PCE) and isolated compounds 3-6 of U. metrosideros on PBMC cytokine secretion. Secreted IFN-γ, IL-17A, IL-8 and TNF cytokines were quantified by flow cytometry and are shown as % of stimulation concentrations of the P/I or CD3/CD28 condition (100%, red dotted line) for each donor. Bar plots show mean normalised cytokine expression of each experimental group (±SE); each point represents the donor mean of quadruplicate samples. Statistical significance was calculated using one sample T-test by comparing stimulation group to each treatment or control. ****=p<0.0001; ***=p<0.001; **=p<0.01; *=p<0.05; no value=p>0.05.

### DETAILED DESCRIPTION OF THE INVENTION

### Definitions

Unless defined otherwise, all technical and scientific terms used herein have the same meaning as commonly understood by those of ordinary skill in the art of the invention. Although any methods and materials similar or equivalent to those described herein can be used in the practice or testing of the present invention, preferred methods and materials are described. For the purposes of the present invention, a number of terms are defined throughout.

The present disclosure may refer to the contents of certain documents being incorporated herein by reference. In the event of any inconsistent teaching between the teaching of the present disclosure and the contents of those documents, the teaching of the present disclosure takes precedence.

It is to be understood that if any prior art publication is referred to herein, such reference does not constitute an admission that the publication forms a part of the common general knowledge in the art in Australia or in any other country.

As used herein, the term “and/or”, e.g., “A and/or B” shall be understood to mean either “A and B” or “A or B” and shall be taken to provide explicit support for both meanings or for either meaning.

As used herein, the term about, unless stated to the contrary, refers to ±10%, of the designated value.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

As used herein, the word “comprise” and other forms of the word, such as “comprising” and “comprises,” means including but not limited to, and is not intended to exclude, for example, other additives, components, integers, or steps.

Each embodiment of the present disclosure described herein is to be applied mutatis mutandis to each and every other embodiment unless specifically stated otherwise or required otherwise by context.

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” (which may be denoted “C₁-C₄ alkyl”) group includes alkyl groups having from 1 to 4 carbons, consisting of 1 carbon atom, 2 carbon atoms, 3 carbon atoms and 4 carbon atoms, e.g. CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

Terms written as “groupAgroupB” are intended to refer to a groupA when linked by a divalent form of groupB. For example, “alkoxy” (which may be denoted “OCₐ—C_{b} alkyl”) denotes alkyl, as herein defined, when linked by an oxygen atom.

As used herein, the term “alkyl” refers to a straight or branched hydrocarbon chain that is fully saturated (i.e., contains no double or triple bonds). An alkyl group may for example have from 1 to 10 carbon atoms which may be denoted “C₁-C₁₀ alkyl”. Typical alkyl groups include, but are not way limited to, methyl, ethyl, propyl, isopropyl, butyl, isobutyl, tertiary butyl, pentyl, hexyl, and the like.

As used herein, the term “alkenyl” means an aliphatic hydrocarbon group containing at least one carbon-carbon double bond and which may be straight or branched. An alkenyl group may have from about 2 to about 10 carbon atoms which may be denoted “C₂-C₁₀ alkenyl”. Exemplary alkenyl groups include ethenyl, propenyl, n-butenyl, and i-butenyl.

As used herein, the term “alkynyl” means an aliphatic hydrocarbon group containing at least one carbon-carbon triple bond and which may be straight or branched. An alkynyl group may have from about 2 to about 10 carbon atoms which may be denoted “C₂-C₁₀ alkynyl”. Examples include ethynyl, 1-propynyl, 2-propynyl, and butynyl isomers, and pentynyl isomers.

As used herein, the terms “halo” or “halogen,” mean, in the context of the compounds defined herein, a fluorine, chlorine, bromine, or iodine atom, unless otherwise dictated by context.

As used herein, the term “pyranose” means a monosaccharide in the form of a 6-membered ring, the 6-membered ring consisting of 6 carbon atoms and one oxygen atom, i.e. a substituted tetrahydropyran.

As used herein, the term “alkylene” means a linear or branched saturated divalent hydrocarbon radical. For example, a C₁-C₁₀ alkylene includes methylene, ethylene, propylene, 1-methylpropylene, 2-methylpropylene, butylene, pentylene, and the like.

As used herein, the term “alkenylene,” means, unless otherwise stated, a divalent radical derived from an alkenyl group as defined above. Similarly, as used herein, the term “alkynylene,” means, unless otherwise stated, a divalent radical derived from an alkynyl group as defined above.

As used herein, the term “oxo” (which may be denoted “═O”) means an oxygen that is double bonded to a carbon atom.

As used herein, the term “hydroxyl” and denotation “OH” are synonymous.

Where the compounds disclosed herein have at least one chiral center, they may exist as individual enantiomers and diastereomers or as mixtures of such isomers, including racemates. Other forms of isomerism include double bond isomerism in which compounds containing a carbon-carbon double bond may exist as Z or E isomers, conformational isomerism, and atropisomerism. Unless otherwise indicated, all such isomers and mixtures thereof are included in the scope of the compounds disclosed herein. Separation of individual isomers or selective synthesis of individual isomers is accomplished by application of various methods which are known to practitioners in the art.

The skilled artisan will also recognize that some structures described herein may be resonance forms or tautomers of compounds that may be fairly represented by other chemical structures. For example, the term “tautomers” may refer to a set of compounds that have the same number and type of atoms but differ in bond connectivity and are in equilibrium with one another. A “tautomer” is a single member of this set of compounds. Typically, a single tautomer is drawn but understood that this single structure may represent all possible tautomers that may exist. Examples may include enol-ketone tautomerism. When a ketone is drawn it may be understood that both the enol and ketone forms are part of the disclosure. Resonance forms and tautomers of compounds are within the scope of the present disclosure.

An isotope of an element other than the most commonly occurring isotope may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise. Such isotopically labelled compounds may for example be useful as research or diagnostic tools.

Those skilled in the art will appreciate that many organic compounds can form complexes in solvents. These complexes are known as “solvates”. For example, a complex with water is known as a “hydrate”. Solvates such as hydrates exist when the compound incorporates solvent. It will be understood that the compounds of the present disclosure, as well as salts thereof, may be present in the form of solvates. Solvates of the compounds which are suitable are those where the associated solvent is pharmaceutically acceptable. Suitable solvates are pharmaceutically acceptable solvates including hydrates. It will be understood that the present disclosure encompasses unsolvated forms of the compounds, as well as solvated forms, such as hydrates.

Compounds disclosed herein may exist in one or more crystalline or amorphous forms. It will be understood that all such forms of the compounds are within the scope of the present disclosure.

As used herein, the term “subject” means an organism that is susceptible to a disease or condition. For example, the subject can be an animal, a mammal, a primate, a livestock animal (e.g., sheep, cow, horse, pig), a companion animal (e.g., dog, cat), or a laboratory animal (e.g., mouse, rabbit, rat, guinea pig, hamster). In some embodiments, the subject is a mammal. In some embodiments, the subject is human.

As used herein, the term “treating” includes curing a disease or disorder, as well as alleviation or reduction of symptoms associated with a disease or disorder. The term treating also includes slowing the progression of a disease or disorder.

As used herein, the term “prevention” includes prophylaxis, and includes reducing the likelihood of contracting a disease or disorder or a symptom thereof.

As used herein in context of treatment or prevention using compounds as described herein, an “effective amount” is an amount of a compound that when administered to a subject, either in a single dose or as part of a series, is effective for treating or preventing a disease or disorder.

As used herein, the term “pharmaceutically acceptable salt” refers to salts which are toxicologically safe for administration to a subject in the course of treating or preventing a disease or disorder.

Compounds as described herein may be isolated from a plant or plant part (e.g. leaves, and the like). As used herein, the term “isolated” means separated from the environment in which it naturally occurs.

### Compounds of Formula (I)

Provided is a compound of formula (I):

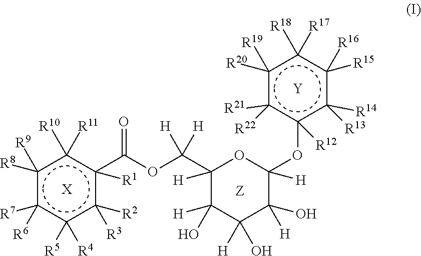

or a pharmaceutically acceptable salt thereof, wherein:
   X is cyclohexane, cyclohexene, cyclohexadiene, or benzene, wherein;
      when X is benzene R¹, R³, R⁵, R⁷, R⁹ and R¹¹ are absent;
      when X is cyclohexene, either: R¹ and R³ are absent and R¹C—CR²R³ is taken to be C═CR²; R³ and R⁵ are absent and R²R³C—CR⁴R⁵ is taken to be R²C═CR⁴; R⁵ and R⁷ are absent and R⁴R⁵C—CR⁶R⁷ is taken to be R⁴C═CR⁶; R⁷ and R⁹ are absent and R⁶R⁷C—CR⁸R⁹ is taken to be R⁶C═CR⁸; R⁹ and R¹¹ are absent and R⁸R⁹C—CR¹⁰R¹¹ is taken to be R⁸C═CR¹⁰; or R¹¹ and R¹ are absent and R¹⁰R¹¹C—CR¹ is taken to be R¹⁰C═C;
      when X is cyclohexadiene, either: R¹, R³, R⁵ and R⁷ are absent and R¹C—CR²R³ and R⁴R⁵C—CR⁶R⁷ are taken to be C═CR² and R⁴C═CR⁶; R¹, R³, R⁷ and R⁹ are absent and R¹C—CR²R³ and R⁶R⁷C—CR⁸R⁹ are taken to be C═CR² and R⁶C═CR⁸; R¹, R³, R⁹ and R¹¹ are absent and R¹C—CR²R³ and R⁸R⁹C—CR¹⁰R¹¹ are taken to be C═CR² and R⁸C═CR¹⁰; R³, R⁵, R⁷ and R⁹ are absent and R²R³C—CR⁴R⁵ and R⁶R⁷C—CR⁸R⁹ are taken to be R²C═CR⁴ and R⁶C═CR⁸; R³, R⁵, R⁹ and R¹¹ are absent and R²R³C—CR⁴R⁵ and R⁸R⁹C—CR¹⁰R¹¹ are taken to be R²C═CR⁴ and R⁸C═CR¹⁰; R³, R⁵, R¹¹ and R¹ are absent and R²R³C—CR⁴R⁵ and R¹⁰R¹¹C—CR¹ are taken to be R²C═CR and R¹⁰C═C; R⁵, R⁷, R⁹ and R¹¹ are absent and R⁴R⁵C—CR⁶R⁷ and R⁸R⁹C—CR¹⁰R¹¹ are taken to be R⁴C═CR⁶ and R⁸C═CR¹⁰, R⁵, R⁷, R¹¹ and R¹ are absent and R⁴R⁵C—CR⁶R⁷ and R¹⁰R¹¹C—CR¹ are taken to be R⁴C═CR⁶ and R¹⁰C═C; or R⁷, R⁹, R¹¹ and R¹ are absent and R⁶R⁷C—CR⁸R⁹ and R¹⁰R¹¹C—CR¹ are taken to be R⁶C═CR⁸ and R¹⁰C═C;
   Y is cyclohexane, cyclohexene, cyclohexadiene, or benzene, wherein;
      when Y is benzene R¹², R¹⁴, R¹⁶, R¹⁸, R²⁰ and R² are absent;
      when Y is cyclohexene, either: R¹² and R¹⁴ are absent and R¹²C—CR¹³R¹⁴ is taken to be C═CR¹³; R¹⁴ and R¹⁶ are absent and R¹³R¹⁴C—CR¹⁵R¹⁶ is taken to be R¹³C═CR¹⁵; R¹⁶ and R¹⁸ are absent and R¹⁵R¹⁶C—CR¹⁷R¹⁸ is taken to be R¹⁵C═CR¹⁷: R¹⁸ and R²⁰ are absent and R¹⁷R¹⁸C—CR¹⁹R²⁰ is taken to be R¹⁷C═CR¹⁹; R²⁰ and R²² are absent and R¹⁹R²⁰C—CR²¹R²² is taken to be R¹⁹C═CR²¹; or R²² and R¹² are absent and R²¹R²²C—CR¹² is taken to be R²¹C═C;
      when Y is cyclohexadiene, either: R¹², R¹⁴, R¹⁶ and R¹⁸ are absent and R¹²C—CR¹³R¹⁴ and R¹⁵R¹⁶C—CR¹⁷R¹⁸ are taken to be C═CR¹³ and R¹⁵C═CR¹⁷; R¹², R¹⁴, R¹⁸ and R²⁰ are absent and R¹²C—CR¹³R¹⁴ and R¹⁷R¹⁸C—CR¹⁹R²⁰ are taken to be C═CR¹³ and R¹⁷C═CR¹⁹; R¹², R¹⁴, R²⁰ and R²² are absent and R¹²C—CR¹³R¹⁴ and R¹⁹R²⁰C—CR²¹R²² are taken to be C═CR¹³ and R¹⁹C═CR²¹; R¹⁴, R¹⁶, R¹⁸ and R²⁰ are absent and R¹³R¹⁴C—CR¹⁵R¹⁶ and R¹⁷R¹⁸C—CR¹⁹R²⁰ are taken to be R¹³C═CR¹⁵ and R¹⁷C═CR¹⁹; R¹⁴, R¹⁶, R²⁰ and R²² are absent and R¹³R¹⁴C—CR¹⁵R¹⁶ and R¹⁹R²⁰C—CR²¹R²² are taken to be R¹³C═CR¹⁵ and R¹⁹C═CR²¹; R¹⁴, R¹⁶, R²² and R¹² are absent and R¹³R¹⁴C—CR¹⁵R¹⁶ and R²¹R²²C—CR¹² are taken to be R¹³C═CR¹⁵ and R²¹C═C; R¹⁶, R¹⁸, R²⁰ and R¹² are absent and R¹⁵R¹⁶C—CR¹⁷R¹⁸ and R¹⁹R²⁰C—CR²¹R²² are taken to be R¹⁵C═CR¹⁷ and R¹⁹C═CR²¹; R¹⁶, R¹⁸, R²² and R¹² are absent and R¹⁵R¹⁶C—CR¹⁷R¹⁸ and R²¹R²²C—CR¹² are taken to be R¹⁵C═CR¹⁷ and R²¹C═C; or R¹⁸, R²⁰, R²² and R¹² are absent and R¹⁷R¹⁸C—CR¹⁹R²⁰ and R²¹R²²C—CR¹² are taken to be R¹⁷C═CR¹⁹ and R²¹C═C;
   at least one of R¹² to R²² is —C₁-C₁₀ alkyl;
   each of R¹ to R²² when present is otherwise independently selected from the group consisting of —H, —OH, halogen, —CN, —SH, —C₁-C₁₀ alkyl, —C₂-C₁₀ alkenyl, —C₂-C₁₀ alkynyl, —C(O)C₁-C₁₀ alkyl, —C(O)C₂-C₁₀ alkenyl, —C(O)C₂-C₁₀ alkynyl, —OC₁-C₁₀ alkyl, —OC₂-C₁₀alkenyl, —OC₂-C₁₀ alkynyl, —OC(O)C₁-C₁₀ alkyl, —OC(O)C₂-C₁₀ alkenyl, —OC(O)C₂-C₁₀ alkynyl, —OC(O)NHC₁-C₁₀ alkyl, —OC(O)NHC₂-C₁₀ alkenyl, —OC(O)NHC₂-C₁₀ alkynyl, and —N(R²³)₂, or one or more of R¹³ and R¹⁴, R¹⁵ and R¹⁶, R¹⁷ and R¹⁸, R¹⁹ and R²⁰, and R²¹ and R², taken together, are ═O, ═S, or ═NR² except when Y is cyclohexane in which case not both of R¹⁵ and R¹⁶, and R¹⁹ and R²⁰, taken together, are ═O; and
   each R²³ is independently selected from the group consisting of H and —C₁-C₁₀ alkyl.

### Z Ring

Z is the tetrahydropyran motif of a pyranose. As such, Z may be said to represent a pyranose. The individual chiral centres of the pyranose may each be independently selected from the (R) and (S) variants. For example, the pyranose may be α- or β-, D- or L-, glucose, allose, altrose, mannose, galactose, talose, iodose or gulose. In preferred embodiments, the pyranose is α- or β-, D- or L-, glucose, mannose or galactose, preferably α- or β-, D- or L-glucose, preferably α- or β-D-glucose, most preferably β-D-glucose.

The compounds of Formula (I) are pyranosides in that the pyranose is substituted at the 1-O-position. In compounds of formula (I), the pyranose is substituted at the 1-O-position with a 6-membered carbocyclic (i.e. “Y”) group. In compounds of formula (I), the pyranose is also substituted at the 6-O-position with a 6-membered carbocyclic (i.e. “X”)-carbonyl-group.

### X Ring

In preferred embodiments, each of R¹ to R¹¹ when present is independently selected from the group consisting of —H, —OH, —C₁-C₁₀ alkyl and —C(O)C₁-C₁₀ alkyl, or one or more of R² and R³, R⁴ and R⁵, R⁶ and R⁷, R⁸ and R⁹, and R¹⁰ and R¹¹, taken together, are ═O. When ═O is present, preferably R⁶ and R⁷ taken together are ═O. Preferably, each of R¹ to R¹¹ when present is independently selected from the group consisting of —H, —OH, —C₁-C₁₀ alkyl and —C(O)C₁-C₁₀ alkyl, preferably —H and —OH.

Preferably, of R¹ to R¹¹, the groups specified to be —C₁-C₁₀ groups are —C₁-C₆ groups, more preferably —C₁-C₄ groups. Preferably, of R¹ to R¹¹, the groups specified to be —C₂-C₁₀ groups are —C₂-C₆ groups, more preferably —C₂-C₄ groups.

In more preferred embodiments, three of R² to R¹¹ are —OH, and each of the remainder of R¹ to R¹¹ is absent or —H. Preferably, one of R⁴ and R⁵, one of R⁶ and R⁷, and one of R⁸ and R⁹ is —OH, and each of the remainder of R¹ to R¹¹ is absent or —H.

When X is cyclohexene, it is preferred that R³ and R⁵ are absent and R²R³C—CR⁴R⁵ is taken to be R²C═CR⁴, R⁵ and R⁷ are absent and R⁴R⁵C—CR⁶R⁷ is taken to be R⁴C═CR⁶, R⁷ and R⁹ are absent and R⁶R⁷C—CR⁸R⁹ is taken to be R⁶C═CR⁸, or R⁹ and R¹¹ are absent and R⁸R⁹C—CR¹⁰R¹¹ is taken to be R⁸C═CR¹⁰.

When X is cyclohexadiene, it is preferred that R³, R⁵, R⁹ and R¹¹ are absent and R²R³C—CR⁴R⁵ and R⁸R⁹C—CR¹⁰R¹¹ are taken to be R²C═CR⁴ and R⁸C═CR¹⁰.

In preferred embodiments, X is benzene. Preferably each of R², R⁴, R⁶, R⁸ and R¹⁰ are independently selected from the group consisting of —H and —OH. Preferably three of R², R⁴, R⁹, R⁸ and R¹⁰ are —OH and each of the remainder is —H. Preferably, R² and R¹⁰ are —H and R⁴, R⁶ and R⁸ are —OH.

### Y Ring

Of Y, at least one of R¹² to R²² is —C₁-C₁₀ alkyl. In preferred embodiments, at least one of R¹³, R¹⁴, R¹⁷, R¹⁸, R²¹ and R²² is —C₁-C₁₀ alkyl. In preferred embodiments, the at least one —C₁-C₁₀ alkyl is methyl.

In preferred embodiments, each of R² to R²² when present is otherwise independently selected from the group consisting of —H, —OH, —C₁-C₁₀ alkyl and —C(O)C₁-C₁₀ alkyl, or one or more of R¹³ and R¹⁴, R¹⁵ and R¹⁶, R¹⁷ and R¹⁸, R¹⁹ and R²⁰, and R²¹ and R²², taken together, are ═O, subject to the proviso that when Y is cyclohexane not both of R¹⁵ and R¹⁶, and R¹⁹ and R²⁰, taken together, are ═O. When ═O is present, preferably one of R¹³ and R¹⁴, R¹⁵ and R¹⁶, R¹⁷ and R¹⁸, R¹⁹ and R²⁰, and R²¹ and R²², taken together, are ═O, preferably wherein R¹⁷ and R¹⁸ taken together are ═O.

In more preferred embodiments, each of R¹², R¹⁵, R¹⁶, R¹⁹ and R²⁰ when present is independently selected from the group consisting of —H, —OH, —C₁-C₁₀ alkyl and —C(O)C₁-C₁₀ alkyl, preferably —H and —OH. Preferably, each of R¹³, R¹⁴, R¹⁷, R¹⁸, R²¹ and R²² when present is independently selected from the group consisting of —H, —OH, —C₁-C₁₀ alkyl and —C(O)C₁-C₁₀ alkyl, preferably —H, —C₁-C₁₀ alkyl and —C(O)C₁-C₁₀ alkyl, or R¹⁷ and R¹⁸ taken together are ═O, at least one of R¹³, R¹⁴, R¹⁷, R¹⁸, R²¹ and R²² being —C₁-C₁₀ alkyl. Preferably, R¹² is absent or selected from —H and —OH. Preferably, at least one of R¹⁷ and R¹⁸ is —C₁-C₁₀ alkyl and the other is absent or —H, or R¹⁷ and R¹⁸ taken together are ═O in which case at least one of R¹³, R¹⁴, R²¹ and R²² is —C₁-C₁₀ alkyl.

When Y is cyclohexene, it is preferred that R¹⁴ and R¹⁶ are absent and R¹³R¹⁴C—CR¹⁵R¹⁶ is taken to be R¹³C═CR¹⁵, R¹⁶ and R¹⁸ are absent and R¹⁵R¹⁶C—CR¹⁷R¹⁸ is taken to be R¹⁵C═CR¹⁷, R¹⁸ and R²⁰ are absent and R¹⁷R¹⁸C—CR¹⁹R²⁰ is taken to be R¹⁷C═CR¹⁹, or R²⁰ and R²² are absent and R¹⁹R²⁰C—CR²¹R²² is taken to be R¹⁹C═CR²¹.

When Y is cyclohexadiene, it is preferred that R¹², R¹⁴, R¹⁸ and R²⁰ are absent and R¹²C—CR¹³R¹⁴ and R¹⁷R¹⁸C—CR¹⁹R²⁰ are taken to be C═CR¹³ and R¹⁷C═CR¹⁹, R¹⁴, R¹⁶, R²⁰ and R²² are absent and R¹³R¹⁴C—CR¹⁵R¹⁶ and R¹⁹R²⁰C—CR²¹R²² are taken to be R¹³C═CR¹⁵ and R¹⁹C═CR²¹, or R¹⁶, R¹⁸, R²² and R¹² are absent and R¹⁵R¹⁶C—CR¹⁷R¹⁸ and R²¹R²²C—CR¹² are taken to be R¹⁵C═CR¹⁷ and R²¹C═C.

In preferred embodiments, Y is cyclohexane, cyclohexene, or benzene.

Specific preferred embodiments include:
a. Y is cyclohexane, R¹³, R¹⁴, R¹⁷, R¹⁸, R²¹ and R²² are independently selected from the group consisting of —H and —C₁-C₁₀ alkyl, at least one of which being —C₁-C₁₀ alkyl, and R¹², R¹⁵, R¹⁶, R¹⁹ and R²⁰ are independently selected from the group consisting of —H and —OH, more preferably wherein three of R¹³, R¹⁴, R²¹ and R²² are —C₁-C₁₀ alkyl and the other is —H, one of R¹⁷ and R¹⁸ is —C₁-C₁₀ alkyl and the other is —H, one of R¹⁵ and R¹⁶ is —OH and the other is —H, one of R¹⁹ and R²⁰ is —OH and the other is —H, and R¹² is H.
b. Y is cyclohexene, R¹⁶ and R¹⁸ are absent and R¹⁵R¹⁶C—CR¹⁷R¹⁸ is taken to be R¹⁵C═CR¹⁷, R¹⁷ is —C₁-C₁₀ alkyl, preferably methyl, and the remainder of R¹² to R²² are —H.
c. Y is cyclohexene, R¹⁴ and R¹⁶ are absent and R¹³R¹⁴C—CR¹⁵R¹⁶ is taken to be R¹³C═CR¹⁵, or R²⁰ and R²² are absent and R¹⁹R²⁰C—CR²¹R²² is taken to be R¹⁹C═CR²¹, R¹⁷ and R¹⁸ taken together are ═O, R¹² is selected from the group consisting of —H and —OH, at least one of R¹³, R¹⁴, R²¹ and R²² is —C₁-C₁₀ alkyl, and the remainder of R¹² to R²² are —H, preferably wherein three of R¹³, R¹⁴, R²¹ and R²² are —C₁-C₁₀ alkyl.
d. Y is benzene, R¹³ and R²¹ are independently selected from the group consisting of —C(O)C₁-C₁₀ alkyl and —C₁-C₁₀ alkyl, R¹⁵ and R¹⁹ are —OH, and R¹⁷ is —C₁-C₁₀ alkyl, more preferably wherein R¹³ is C(O)C₁-C₁₀ alkyl, R¹⁵ and R¹⁹ are —OH, and R¹⁷ is —C₁-C₁₀ alkyl.

In specific more preferred embodiments, Y is cyclohexane or benzene. Preferably, of R¹² to R²², at least two are —C₁-C₁₀ alkyl and two are —OH. Preferably, R¹² is absent or —H, one of R¹⁵ and R¹⁶ and one of R¹⁹ and R²⁰ is —OH and the other is absent or —H, one of R¹⁷ and R¹⁸ is —C₁-C₁₀ alkyl, preferably methyl, and the other is absent or H, one of R¹³ and R¹⁴ is —C₁-C₁₀ alkyl or —C(O)C₁-C₁₀ alkyl and the other is absent or —H, and of R²¹ and R²² is —C₁-C₁₀ alkyl and the other is —C₁-C₁₀ alkyl, absent or —H.

Preferably, of R¹² to R²², the groups specified to be —C₁-C₁₀ groups are —C₁-C₆ groups, more preferably —C₁-C₄ groups. Preferably, of R² to R²², the groups specified to be —C₂-C₁₀ groups are —C₂-C₆ groups, more preferably —C₂-C₄ groups.

### Preferred Combinations

Preferably, X is benzene in which case R¹, R³, R⁵, R⁷, R⁹ and R¹¹ are absent, and Y is cyclohexane, cyclohexene, or benzene.

Preferably, each of R¹ to R¹¹ when present is independently selected from the group consisting of —H, —OH, —C₁-C₁₀ alkyl and —C(O)C₁-C₁₀ alkyl, preferably —H and —OH, and each of R¹² to R²² when present is otherwise independently selected from the group consisting of —H, —OH, —C₁-C₁₀ alkyl and —C(O)C₁-C₁₀ alkyl, or one or more of R¹³ and R¹⁴, R¹⁵ and R¹⁶, R¹⁷ and R¹⁸, R¹⁹ and R²⁰, and R²¹ and R², taken together, are ═O, subject to the proviso that when Y is cyclohexane not both of R¹¹ and R¹⁶, and R¹⁹ and R²⁰, taken together, are ═O. When ═O is present, preferably one of R¹³ and R¹⁴, R¹⁵ and R¹⁶, R¹⁷ and R¹⁸, R¹⁹ and R²⁰, and R²¹ and R²², taken together, are ═O, preferably wherein R¹⁷ and R¹⁸ taken together are ═O.

Preferably, three of R³, R⁵, R⁷, R⁹ and R¹¹ are —OH, and each of the remainder is —H, each of R¹², R¹⁵, R¹⁶, R¹⁹ and R²⁰ when present is independently selected from the group consisting of —H, —OH, —C₁-C₁₀ alkyl and —C(O)C₁-C₁₀ alkyl, preferably —H and —OH, and the remainder of R¹² to R²² is absent or independently selected from the group consisting of —H, —OH, —C₁-C₁₀ alkyl and —C(O)C₁-C₁₀ alkyl, preferably —H, —C₁-C₁₀ alkyl and —C(O)C₁-C₁₀ alkyl, or R¹⁷ and R¹⁸ taken together are ═O.

Preferably, each of R⁴, R⁶ and R⁸ is —OH, and each of R² and R¹⁰ is —H, R¹² is absent or selected from —H and —OH, at least one of R¹⁷ and R¹⁸ is —C—C₀ alkyl and the other is absent or —H, or R¹⁷ and R¹⁸ taken together are ═O, and the remainder of R¹² to R²² is absent or independently selected from the group consisting of —H, —OH, —C₁-C₁₀ alkyl and —C(O)C₁-C₁₀ alkyl.

In specific preferred embodiments, X is benzene, each of R⁴, R⁶ and R⁸ is —OH, and each of R² and R¹⁰ is —H, and Y is as described above in respect of its specific preferred embodiments.

In most preferred embodiments, the compound of Formula (I) is selected from the group consisting of:

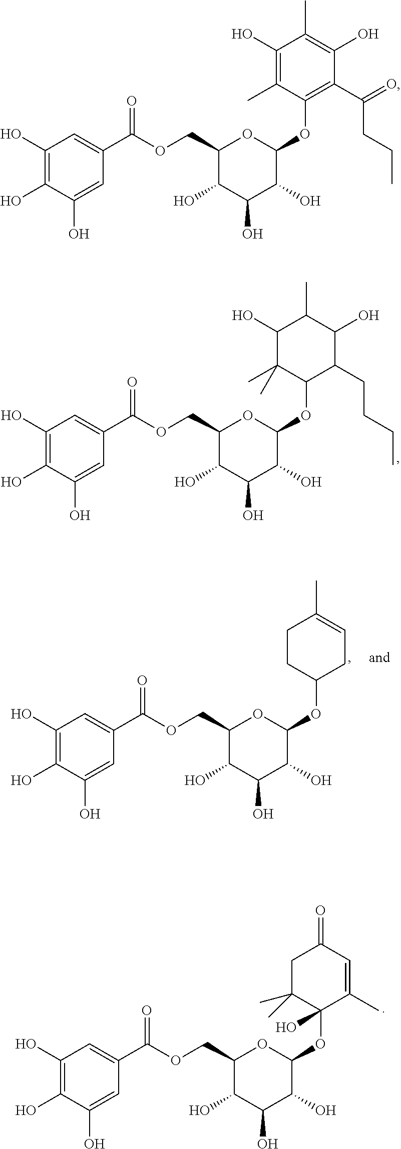 [SMILES: CCCC(=O)c1c(O)c(C)c(O)c(C)c1O[C@@H]1O[C@H](COC(=O)c2cc(O)c(O)c(O)c2)[C@@H](O)[C@H](O)[C@H]1O ; CCCCC1C(O)C(C)C(O)C(C)(C)C1O[C@@H]1O[C@H](COC(=O)c2cc(O)c(O)c(O)c2)[C@@H](O)[C@H](O)[C@H]1O ; CC1=CCC(O[C@@H]2O[C@H](COC(=O)c3cc(O)c(O)c(O)c3)[C@@H](O)[C@H](O)[C@H]2O)CC1 ; CC1=CC(=O)CC(C)(C)[C@@]1(O)O[C@@H]1O[C@H](COC(=O)c2cc(O)c(O)c(O)c2)[C@@H](O)[C@H](O)[C@H]1O]

In some embodiments, the compound of formula (I) is not a salt. In other embodiments, the compound of formula (I) is provided in the form of a salt, preferably a pharmaceutically acceptable salt. Suitable salts include those formed with organic or inorganic acids or bases. Exemplary salts and methods for forming salts of organic compounds are known to those of skill in the art.

### Compounds of Formula (II)

Described herein is a compound of Formula (II):

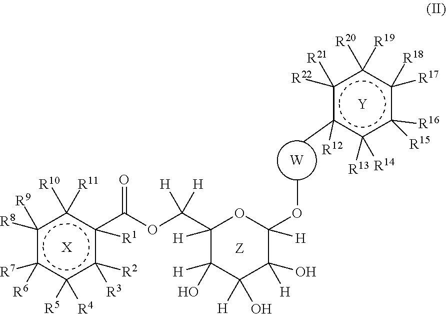

or a pharmaceutically acceptable salt thereof, wherein:
   X is cyclohexane, cyclohexene, cyclohexadiene, or benzene, wherein;
   when X is benzene R¹, R³, R⁵, R⁷, R⁹ and R¹¹ are absent;
   when X is cyclohexene, either: R¹ and R³ are absent and R¹C—CR²R³ is taken to be C═CR²; R³ and R⁵ are absent and R²R³C—CR⁴R⁵ is taken to be R²C═CR⁴; R⁵ and R⁷ are absent and R⁴R⁵C—CR⁶R⁷ is taken to be R⁴C═CR⁶; R⁷ and R⁹ are absent and R⁶R⁷C—CR⁸R⁹ is taken to be R⁶C═CR⁸; R⁹ and R¹¹ are absent and R⁸R⁹C—CR¹⁰R¹¹ is taken to be R⁸C═CR¹⁰; or R¹¹ and R are absent and R¹⁰R¹¹C—CR¹ is taken to be R¹⁰C═C;
   when X is cyclohexadiene, either: R¹, R³, R⁵ and R⁷ are absent and R¹C—CR²R³ and R⁴R⁵C—CR⁶R⁷ are taken to be C═CR² and R⁴C═CR⁶; R¹, R³, R⁷ and R⁹ are absent and R¹C—CR²R³ and R⁶R⁷C—CR⁸R⁹ are taken to be C═CR² and R⁶C═CR⁸; R¹, R³, R⁹ and R¹¹ are absent and R¹C—CR²R³ and R⁸R⁹C—CR¹⁰R¹¹ are taken to be C═CR² and R⁸C═CR¹⁰; R³, R⁵, R⁷ and R⁹ are absent and R²R³C—CR⁴R⁵ and R⁶R⁷C—CR⁸R⁹ are taken to be R²C═CR⁴ and R⁶C═CR⁸; R³, R⁵, R⁹ and R¹¹ are absent and R²R³C—CR⁴R⁵ and R⁸R⁹C—CR¹⁰R¹¹ are taken to be R²C═CR⁴ and R⁸C═CR¹⁰; R³, R⁵, R¹¹ and R¹ are absent and R²R³C—CR⁴R⁵ and R¹⁰R¹¹C—CR¹ are taken to be R²C═CR and R¹⁰C═C; R⁵, R⁷, R⁹ and R¹¹ are absent and R⁴R⁵C—CR⁶R⁷ and R⁸R⁹C—CR¹⁰R¹¹ are taken to be R⁴C═CR⁶ and R⁸C═CR¹⁰; R⁵, R⁷, R¹¹ and R¹ are absent and R⁴R⁵C—CR⁶R⁸ and R¹⁰R¹¹C—CR¹ are taken to be R⁴C═CR⁶ and R¹⁰C═C; or R⁷, R⁹, R¹¹ and R¹ are absent and R⁶R⁷C—CR⁸R⁹ and R¹⁰R¹¹C—CR¹ are taken to be R⁶C═CR⁸ and R¹⁰C═C;
Y is cyclohexane, cyclohexene, cyclohexadiene, or benzene, wherein;
   when Y is benzene R¹², R¹⁴, R¹⁶, R¹⁸, R²⁰ and R²² are absent:
   when Y is cyclohexene, either: R¹² and R¹⁴ are absent and R²C—CR¹³R¹⁴ is taken to be C═CR¹³; R¹⁴ and R¹⁶ are absent and R¹³R¹⁴C—CR¹⁵R¹⁶ is taken to be R¹³C═CR¹⁵; R¹⁶ and R¹⁸ are absent and R¹⁵R¹⁶C—CR¹⁷R¹⁸ is taken to be R¹⁵C═CR¹⁷; R¹⁸ and R²⁰ are absent and R¹⁷R¹⁸C—CR¹⁹R²⁰ is taken to be R¹⁷C═CR¹⁹; R²⁰ and R²² are absent and R¹⁹R²⁰C—CR²¹R²² is taken to be R¹⁹C═CR²¹; or R²² and R¹² are absent and R²¹R²²C—CR¹² is taken to be R²¹C═C;
   when Y is cyclohexadiene, either: R¹², R¹⁴, R¹⁶ and R¹⁸ are absent and R¹²C—CR¹³R¹⁴ and R¹⁵R¹⁶C—CR¹⁷R¹⁸ are taken to be C═CR¹³ and R¹⁵C═CR¹⁷; R¹², R¹⁴, R¹⁸ and R²⁰ are absent and R¹²C—CR¹³R¹⁴ and R¹⁷R¹⁸C—CR¹⁹R²⁰ are taken to be C═CR¹³ and R¹⁷C═CR¹⁹; R¹², R¹⁴, R²⁰ and R²² are absent and R¹²C—CR¹³R¹⁴ and R¹⁹R²⁰C—CR²¹R²² are taken to be C═CR¹³ and R¹⁹C═CR²¹; R¹⁴, R¹⁶, R¹⁸ and R²⁰ are absent and R¹³R¹⁴C—CR¹⁵R¹⁶ and R¹⁷R¹⁸C—CR¹⁹R²⁰ are taken to be R¹³C═CR¹⁵ and R¹⁷C═CR¹⁹; R¹⁴, R¹⁶, R²⁰ and R²² are absent and R¹³R¹⁴C—CR¹⁵R¹⁶ and R¹⁹R²⁰C—CR²¹R²² are taken to be R¹³C═CR¹⁵ and R¹⁹C═CR²¹; R¹⁴, R¹⁶, R²⁰ and R²² are absent and R¹³R¹⁴C—CR¹⁵R¹⁶ and R²¹R²²C—CR¹² are taken to be R¹³C═CR¹⁵ and R²¹C═C; R¹⁶, R¹⁸, R²⁰ and R²² are absent and R¹⁵R¹⁶C—CR¹⁷R¹⁸ and R¹⁹R²⁰C—CR²¹R²² are taken to be R¹⁵C═CR¹⁷ and R¹⁹C═CR²¹; R¹⁶, R¹⁸, R²² and R¹² are absent and R¹⁵R¹⁶C—CR¹⁷R¹⁸ and R²¹R²²C—CR¹² are taken to be R¹⁵C═CR¹⁷ and R²¹C═C; or R¹⁸, R²⁰, R²² and R¹² are absent and R¹⁷R¹⁸C—CR¹⁹R²⁰ and R²¹R²²C—CR¹² are taken to be R¹⁷C═CR¹⁹ and R²¹C═C;
each of R¹ to R²² when present is otherwise independently selected from the group consisting of —H, —OH, halogen, —CN, —SH, —C₁-C₁₀ alkyl, —C₂-C₁₀ alkenyl, —C₂-C₁₀ alkynyl, —C(O)C₁-C₁₀ alkyl, —C(O)C₂-C₁₀ alkenyl, —C(O)C₂-C₁₀ alkynyl, —OC₁-C₁₀ alkyl, —OC₂-C₁₀ alkenyl, —OC₂-C₁₀ alkynyl, —OC(O)C₁-C₁₀ alkyl, —OC(O)C₂-C₁₀ alkenyl, —OC(O)C₂-C₁₀ alkynyl, —OC(O)NHC₁-C₁₀ alkyl, —OC(O)NHC₂-C₁₀ alkenyl, —OC(O)NHC₂-C₁₀ alkynyl, and —N(R²³)₂, or one or more of R¹³ and R¹⁴, R¹⁵ and R¹⁶, R¹⁷ and R¹⁸, R¹⁹ and R²⁰, and R²¹ and R²², taken together, are ═O, ═S, or ═NR²³;
each R²³ is independently selected from the group consisting of H and —C₁-C₁₀ alkyl; and
W is absent or selected from the group consisting of C₁-C₁₀ alkylene, C₂-C₁₀ alkenylene, C₂-C₁₀ alkynylene, C(O)C₁-C₁₀ alkylene, C(O)C₂-C₁₀ alkenylene, and C(O)C₂-C₁₀ alkynylene.

A compound of Formula (I) is encompassed within a compound of Formula (11) when W is absent. The description of a compound of Formula (I) herein is otherwise equally applicable to a compound of Formula (II).

In a compound of formula (II), it is not necessarily required that at least one of R¹² to R²² is —C₁-C₁₀ alkyl or that when Y is cyclohexane not both of R⁵ and R¹, and R¹⁹ and R²⁰, taken together, are ═O, though it is preferred that at least one of R¹² to R²² is —C₁-C₁₀ alkyl, preferably a methyl, and/or that when Y is cyclohexane not both of R¹⁵ and R¹⁶, and R¹⁹ and R²⁰, taken together, are ═O.

Preferably, of W, the groups specified to be —C₁-C₁₀ groups are —C₁-C₆ groups, more preferably —C₁-C₄ groups. Preferably, of W, the groups specified to be —C₂-C₁₀ groups are —C₂-C₆ groups, more preferably —C₂-C₄ groups.

In preferred embodiments, W is absent or selected from the group consisting of C₁-C₄ alkylene and C₂-C₄ alkenylene. In preferred embodiments, when present W is selected from the group consisting of —C(CH₃)₂— and —CH(CH₃)CH═CH—.

In most preferred embodiments, the compound of Formula (II) is selected from the group consisting of:

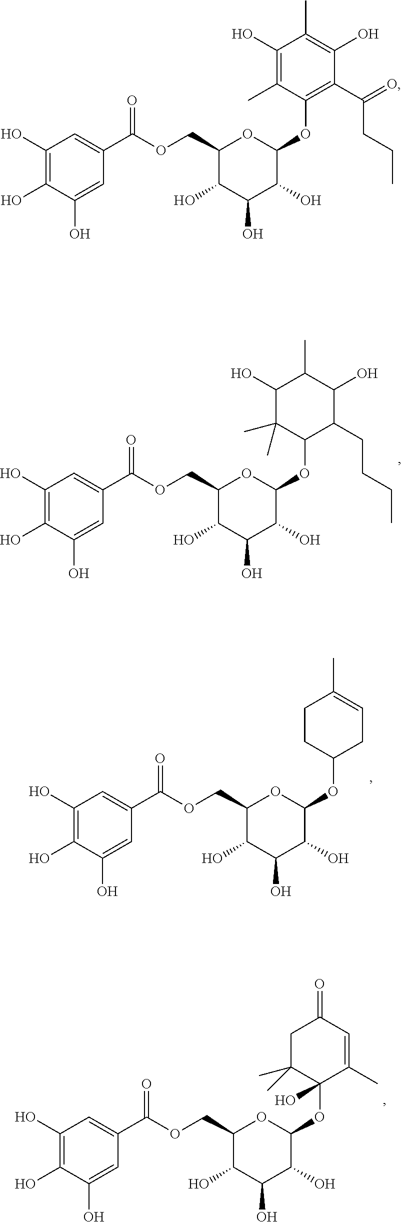 [SMILES: CCCC(=O)c1c(O)c(C)c(O)c(C)c1O[C@@H]1O[C@H](COC(=O)c2cc(O)c(O)c(O)c2)[C@@H](O)[C@H](O)[C@H]1O ; CCCCC1C(O)C(C)C(O)C(C)(C)C1O[C@@H]1O[C@H](COC(=O)c2cc(O)c(O)c(O)c2)[C@@H](O)[C@H](O)[C@H]1O ; CC1=CCC(O[C@@H]2O[C@H](COC(=O)c3cc(O)c(O)c(O)c3)[C@@H](O)[C@H](O)[C@H]2O)CC1 ; CC1=CC(=O)CC(C)(C)[C@@]1(O)O[C@@H]1O[C@H](COC(=O)c2cc(O)c(O)c(O)c2)[C@@H](O)[C@H](O)[C@H]1O]

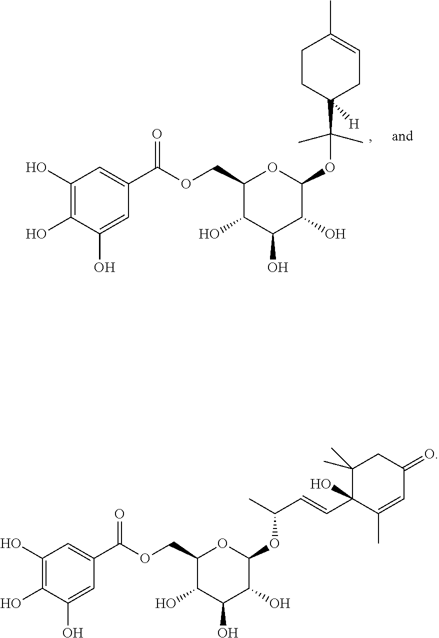

### Compound Preparation

Compounds of Formula (I) or Formula (II) may be obtained by isolation from a plant or plant part, or by derivatisation of the isolated compound, or by derivatisation of a related compound.

The plant may be of the family Myrtaceae, preferably of the genus Uromyrtus or Pimenta and preferably of the species Uromyrtus metrosideros or Pimenta dioica.

Compounds may be isolated by extraction of the plant or plant part. The plant part include may include fruit, seed, bark, leaf, flower, roots and wood. Preferably, compounds are extracted from leaves. Extraction methods are known to those of skill in the art, and generally include pulverisation of plant biomass, solvent extraction, for example with a polar solvent, concentration and separation by chromatographic techniques.

In particular embodiments, the following compounds may be obtained by isolation from the leaves of a plant of the species Uromyrtus metrosideros:

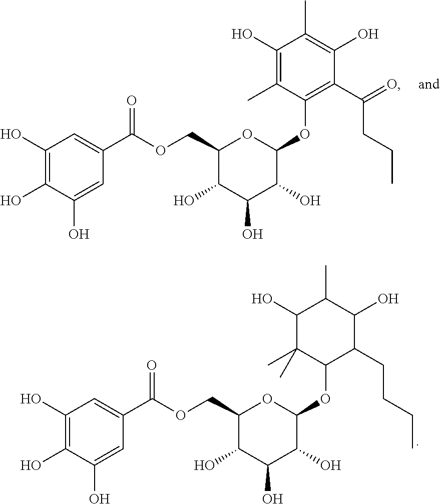 [SMILES: CCCC(=O)c1c(O)c(C)c(O)c(C)c1O[C@@H]1O[C@H](COC(=O)c2cc(O)c(O)c(O)c2)[C@@H](O)[C@H](O)[C@H]1O ; "and" ; CCCCC1C(O)C(C)C(O)C(C)(C)C1O[C@@H]1O[C@H](COC(=O)c2cc(O)c(O)c(O)c2)[C@@H](O)[C@H](O)[C@H]1O]

In particular embodiments, the following compounds may be obtained by isolation from the leaves of a plant of the species Pimenta dioica:

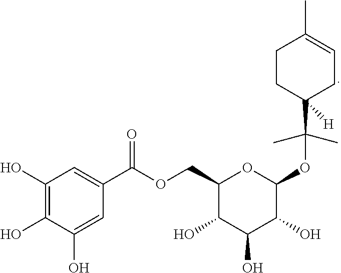

The related compound which may be derivatised to obtain a compound of Formula (I) or Formula (II) may also be obtained by isolation from a plant or plant part, for example a plant of the family Myrtaceae, Apocynaceae, Betulaceae, Vitaceae, Solanaceae, Polygonaceae, Fabaceae or Rosaceae. The plant may be of the genus Catharanthus, Betula, Vitis, Nicotiana, Persicaria, Uromyrtus, Bauhinia, Cydonia, Chaenomeles, Eriobotrya, Pimenta or Callistenon. Preferably, the plant is of the species Catharanthus roseus, Betula pubescens, Vitis vinifera, Nicotiana spp., Persicaria hydropiper, Bauhinia variegata, Cydonia oblonga, Chaenomeles japonica, Eriobotrya japonica, Uromyrtus metrosideros, Pimenta dioica and Callistemon citrinus.

Derivatives of the compounds can be obtained by techniques known in the art.

Compounds of Formula (I) and Formula (II) may also be obtained by chemical synthesis using methods as generally described in the following documents, each of which is incorporated herein in its entirety: H. P. V. R. Ziaullah, Bioorganic Chemistry, 2016, 65 17-25; Zhu, X. et al., Food & Function, 2019, 10, 4189; and Zhao, Y. et al. Bioorganic & Medicinal Chemistry Letters, 2015, 25, 1509-1514.

### Therapeutic Applications

It has been found that compounds of Formula (I) and Formula (II) or salts thereof are biologically active candidates for use in therapy.

Accordingly, the present invention provides a compound of Formula (I) or Formula (II) or pharmaceutically acceptable salt as described herein, or a pharmaceutical composition as described herein, for use in therapy.

In particular, it has been found that compounds of Formula (I) and Formula (II), and pharmaceutical compositions comprising the compounds or salts, have potential for use in preventing or treating inflammatory diseases or disorders. Accordingly, the preferred therapeutic application is in preventing or treating an inflammatory disease or disorder.

Thus, the present invention also provides a method for preventing or treating an inflammatory disease or disorder in a subject, comprising administering to the subject an effective amount of a compound of formula (II) or a pharmaceutically acceptable salt thereof as described herein, including a compound of formula (I) or a pharmaceutically acceptable salt thereof.

Also provided is the use of a compound of Formula (I) or Formula (II) or pharmaceutically acceptable salt as described herein, or a pharmaceutical composition as described herein, in the manufacture of a medicament for preventing or treating an inflammatory disease or disorder.

Without wishing to be limited by theory, it is believed that compound of Formula (I) and Formula (II) have potential for use in preventing or treating inflammatory diseases or disorders through one or more of the following mechanisms of action: (a) direct neutralisation of inflammatory cytokines; and (b) binding of receptors on or inside immune cells and interfering with molecular/biological pathways to inhibit release of inflammatory cytokines by immune cells, and/or to affect additional (bystander) immune cells to reduce or prevent the inflammatory cytokine secretion by bystander immune cells.

Accordingly, in preferred embodiments, the inflammatory disease or disorder is one which is associated with a cytokine, preferably one which is associate with overexpression of a cytokine, and more particularly where the cytokine is an interleukin or an interferon.

Applicable inflammatory diseases or disorders may include inflammatory bowel disease, fatty liver disease, endometriosis, diabetes mellitus (type I and type II), asthma, arthritis including rheumatoid arthritis, Alzhemier's disease, Parkinson's disease, cardiovascular disease, chronic obstructive pulmonary disease, psoriasis, ankylosing spondylitis, antiphospholipid antibody syndrome (APS), gout, myositis, scleroderma, Sjogren's syndrome, lupus, myelitis, encephalitis, meningitis, carditis, enteritis and vasculitis.

In preferred embodiments, the inflammatory disease or disorder is one which is associated with one or more of IFN-γ, IL-17A and IL-8, and preferably overexpression of one or more of IFN-γ, IL-17A and IL-8. IBD is an inflammatory condition which is associated with overexpression all three of IFN-γ, IL-17A and IL-8. Accordingly, in particularly preferred embodiments the inflammatory diseases or disorders is IBD.

In some embodiments, the compound, salt or pharmaceutical composition is used for preventing a disease or disorder. In some other embodiments, the compound, salt or pharmaceutical composition is used for treating a disease or disorder.

The compounds, salts and pharmaceutical compositions can in principle be used for therapy of any subject suffering from an inflammatory disease or disorder. Subjects may include mammalian subjects such as humans, primates, and livestock and companion animals. In preferred embodiments, the subject is a human subject.

The compound, salt or pharmaceutical composition may be administered according to any suitable dosing regimen. The dosing regimen for any particular subject may depend upon a variety of factors including the disease or disorder being treated and its severity. This may involve a variety of administration routes such as oral, nasal, intra-arterial, intravenous or intramuscular administration. This may involve a variety of dosing intervals such as multiple doses daily, one a day, one every other day, or one or more times weekly, fortnightly or monthly, for a fixed or ongoing period of time. This may involve dosing in any suitable amount, which may be for example be in the range of from 1 mg to 500 mg per day, or in the range of from 5 mg to 400 mg, or in the range of from 10 mg to 300 mg, such as for example 50 mg, 100 mg, 150 mg, 200 mg or 250 mg. This may also involve administration as the sole active agent or in combination with another active agent. Example other active agents include nonsteroidal anti-inflammatory drugs, corticosteroids and biologic drugs such as monoclonal antibodies, including abatacept, adalimumab, certolizumab, etanercept, infliximab, golimumab, rituximab, tocilizumab and vedolizumab. When in combination, the other active agent may be administered simultaneously, sequentially or separately. When simultaneously, the other agent may part of the same pharmaceutical composition as the compound of Formula (I) or Formula (II) or salt thereof, or in a separate composition.

### Pharmaceutical Compositions

The compound of formula (I) or (II) or pharmaceutically acceptable salt thereof can be provided in the form of a pharmaceutical composition, e.g. for use in treatment of a disease or disorder as defined herein. There is therefore provided a pharmaceutical composition comprising a compound of Formula (I) or Formula (II) or pharmaceutically acceptable salt as described herein, and one or more pharmaceutically acceptable excipients.

The pharmaceutical composition may be in a variety of different forms for administration by a variety of different routes. For example, it may be in in solid, semi-solid or liquid dosage form, e.g. in the form of a tablet, spray or solution, for example, for oral, nasal, intra-arterial, intravenous or intramuscular administration. The pharmaceutical composition may be provided in unit dosage form, being a composition containing an amount of a compound of Formula (I) or Formula (II) or salt thereof sufficient to provide a single dose or part-single dose of that compound or salt.

The form of the pharmaceutical composition and excipients may depend on the route of administration.

Pharmaceutical compositions may be prepared by conventional methods known to those of skill in the art. The pharmaceutical compositions may contain from about 0.1% to about 99%, from about 5% to about 90%, or from about 1% to about 20% of compound of Formula (I) or Formula (II) or salt thereof, by weight or volume, with the balance being one or more suitable pharmaceutical excipients.

In some embodiments, the pharmaceutical composition is a solid composition.

Excipients in a solid pharmaceutical composition may include one or more fillers, binders, disintegrants, lubricants, preservatives, antioxidants, antimicrobial agents, sweeteners, and the like. Example fillers include celluloses, calcium carbonate, mannitol, lactose and silicon dioxide. Example binders include magnesium aluminum silicate, starches, gelatin, tragacanth, methylcellulose, sodium carboxymethylcellulose, magnesium carbonate and polyvinylpyrrolidone. Example disintegrants include starches, alginic acid, cellulose and derivatives thereof such as microcrystalline cellulose and sodium carboxymethylcellulose, and polyvinylpyrrolidone. Example lubricants include stearic acid and its salts such as magnesium or calcium salts. Example preservatives include benzalkonium chloride, PHMB, chlorobutanol, thimerosal, phenylmercuric, acetate and phenylmercuric nitrate. Example sweeteners include aspartame and saccharin, and sugars such as lactose, dextrose, sucrose, mannitol, sorbitol and glucose.

In some embodiments, the pharmaceutical composition is a liquid composition.

Liquid compositions include solutions, including aqueous solutions, emulsions and suspensions. Excipients in a liquid composition typically contain a solvent and/or diluent (e.g. water and/or an organic solvent and/or an oil) and may contain additional components such as a solubilizing agent, surfactant, emulsifier, preservative, antioxidant, pH adjusting agent, buffer, tonicity modifier, one or more salts and sweeteners. Example solvents/diluents include water and vegetable oils, e.g. cottonseed, groundnut, corn, germ, olive, castor, and sesame oils. Carriers for emulsions and suspensions may include ethanol, glycerol, propylene glycol, polyethylene glycol, liquid sucrose, sorbitol and water. Example emulsifiers include ethyl alcohol, isopropyl alcohol, ethyl carbonate, ethyl acetate, benzyl alcohol, benzyl benzoate and propylene glycol. Example pH adjusting agents may include sodium hydroxide, sodium carbonate, sodium acetate, hydrochloric acid and citric acid.

Other excipients and techniques for making pharmaceutical compositions are known to those of skill in the art.

The compounds of formula (I) or Formula (II) or pharmaceutically acceptable salts thereof may be administered in combination with a further active agent. In some embodiments, the pharmaceutical composition comprising the compound of formula (I) or Formula (II) or pharmaceutically acceptable salt thereof may also contain a further therapeutic agent. Example further therapeutic agents include nonsteroidal anti-inflammatory drugs, corticosteroids and biologic drugs such as monoclonal antibodies, including abatacept, adalimumab, certolizumab, etanercept, infliximab, golimumab, rituximab, tocilizumab and vedolizumab.

In order that the invention may be readily understood and put into practical effect, particular preferred embodiments will now be described by way of the following non-limiting examples.

### EXAMPLES

In the Examples, compounds are isolated from Uromyrtus metrosideros leaf extract. Extracts of U. metrosideros leaves, collected from a mountain in the Australian Wet Tropics World Heritage Area (WTWHA), were subjected to semi-preparative high-performance liquid chromatography (HPLC) separation. Six primary metabolites were identified using mass spectrometry (MS) and nuclear magnetic resonance spectroscopy (NMR). To determine a non-toxic concentration of the compounds for subsequent cytokine screening assays, human PBMCs were monitored for early signs of cytotoxicity (up to 24 h) following exposure to the compounds and polar crude extract (PCE). Compounds were tested for in vitro anti-inflammatory properties against experimentally-activated T cells. For the initial screening, human PBMCs were stimulated with P/I. P/I is an efficient, non-specific method of in vitro T cell stimulation, which bypasses surface membrane receptors to rapidly activate intracellular signalling pathways, including compounds of interest. P/I-activated cells were treated with 10 μg/mL purified compounds, PCE, or cyclosporine (CsA; a common immunosuppressant medication). After 20 h of treatment, the release of four cytokines—IFN-γ, IL-17A, IL-8, and TNF were measured in the cell culture supernatant. Compounds showed inhibition of IFN-γ, IL-17A, and IL-8 secretion from phorbol myristate acetate/ionomycin (P/I) and anti-CD3/anti-CD28-activated human peripheral blood mononuclear cells (PBMC), yet without reducing PBMC viability.

Taken together, the data reveals U. metrosideros-derived and related compounds with promising anti-inflammatory properties, particularly cytokine suppressive properties, as candidates for treatment of inflammatory conditions, including IBD.

### Example 1—Compound Extraction & Isolation

### Plant Material

U. metrosideros leaves and branches were collected during the dry season in June 2020 from Mount Lewis National Park, Queensland, Australia (16° 30 38.88″ S; 145° 16′ 9.84″ E, altitude 1227 m above sea level). A voucher herbarium specimen (Worboys S. J., 2038) was lodged at the Australian Tropical Herbarium (accession number CNS150251.1 dated 20 Aug. 2020).

### Extraction

Fresh plant material (1 kg fresh weight) was washed under running tap water and oven-dried at 40° C. for 5 days. After drying, leaves were removed from woody stems and ground to a fine powder using a blender. Ground leaves (0.6 kg) were extracted thrice for 2 h in analytical grade cold methanol (solvent:tissue, 5:1, v/w), filtered (Whatman filter paper, Whatman Asia Pacific, San Centre, Singapore) and pooled. The methanol extract was concentrated at 40° C. using a rotary evaporator (G5 Heidolf CVC 3000 Vacuubrand, John Morris Scientific), and fractionated using CHCl₃/H₂O biphasic separation process. The polar CHCl₃ fraction containing non-polar components including waxes, oils and chlorophyll was concentrated and stored. The polar fraction was freeze-dried and yielded 52.84 g dwt.

### Isolation

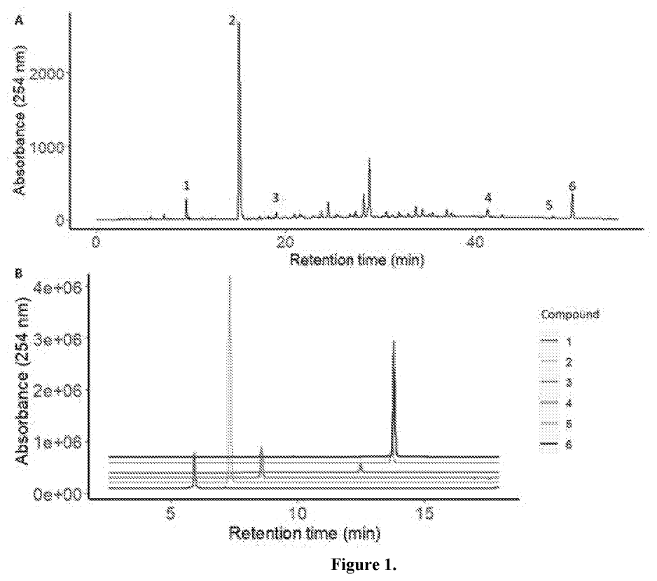

The polar fraction (2.5 g) was suspended in 0.05% (v/v) TFA (40 mg/mL) and centrifuged at 5,000 g for 5 min. The supernatant was filtered (Whatman UNIFLO 25 mm 0.45 μm PES filter media, Whatman Asia Pacific, San Centre, Singapore) and fractionated using an Agilent 1260 HPLC system coupled with a DAD detector (UV 254 nm) and semi-preparative column (Kinetex, C18 10×250 mm) at a flow rate of 5 mL/min. The mobile phases were: A, water with 0.05% (v/v) TFA; and B, 90% acetonitrile (HPLC grade, Fisher Chemical™) with 0.045% (v/v) TFA. The gradient programme was: 0-60 min, linear gradient 2%-40% B; 60-62 min, linear gradient 40%-100% B; 62-67 min isocratic 100% B; 67-70 min, isocratic 2% B. Of the few HPLC chromatogram peaks, six compounds, numbered 1-6 in the order of their fraction elution times (FIG. 1A), were yielded. The purity of these six compounds was determined using analytical HPLC (FIG. 1B).

Fraction purity and compound masses (m/z) were obtained using liquid chromatography-mass spectrometry (LC-MS)—using a Shimadzu LCMS-2020 mass spectrometer coupled to a Shimadzu Prominence HPLC system (Shimadzu, Kyoto, Japan) using an analytical column (Agilent poroshell 120, C18 4.6×150 mm). The mobile phases were: A, water with 0.05% (v/v) FA; and B, 90% acetonitrile (HPLC grade, Fisher Chemical™) with 0.045% (v/v) FA. The gradient programme was 0-15 min, linear gradient 2%-60% B; 15-15.5 min, linear gradient 60%-100% B; 15.5-17.5 min isocratic 100% B; 17.5-18 min linear gradient 100%-2% B; 18-23 min, isocratic 2% B) with a flow rate of 0.8 ml/min. The UV absorbance was observed at 254 nm on a Shimadzu SPD-20A detector. Mass spectra were collected in the negative mode with a scan range of m/z 100-2000, with a detector voltage of 1.15 kV, nebulising gas flow of 1.5 L/min, and drying gas flow of 15 L/min. Data were collected and analysed using the Shimadzu LabSolutions v5.96 software (Shimadzu, Kyoto, Japan). Six pure fractions (compounds 1-6) were collected.

HPLC data were exported from Shimadzu LabSolutions v5.96 software, and chromatograms were plotted in the RStudio v. 1.2.5033, using the ‘ggplot2’, ‘ggpubr’ and ‘tidyverse’ packages.

Table 1 shows the masses and relative abundance within the dried polar crude extract.

TABLE 1
HPLC and mass spectrometry characteristics of compounds 1-6 isolated from Uromyrtus metrosideros crude extract.

| Compound | RT (min) | m/z [M − H]⁻ | MS/MS fragments | MF | RA (%) |
|---|---|---|---|---|---|
| 1 | 10.6 | 313.07 | 235.03, 207.03, 193.02 | C₁₃H₁₄O₉ | 1.4 |
| 2 | 16.6 | 327.08 | 312.05, 249.04, 235.03, 207.03, 194.02 | C₁₄H₁₆O₉ | 12.7 |
| 3 | 19.3 | 385.19 | 223.14, 205.12, 179.06, 167, 161.05, 153.09, 89.02 | C₁₉H₃₀O₈ | 0.2 |
| 4* | 44.5 | 537.16 | 467.13, 313.06, 271.05, 223.10, 169.01, 153.06, 125.02 | C₂₅H₃₀O₁₃ | 1.4 |
| 5 | 51.4 | 467.19 | 423.20, 313.06, 271.05, 223.06, 211.03, 169.01, 151.01, 125.02 | C₂₃H₃₂O₁₀ | 0.2 |
| 6* | 53.2 | 543.24 | 313.06, 271.05, 229.18, 211.03, 169.01, 151.01, 125.02 | C₂₆H₄₀O₁₂ | 2.9 |

m/z represents [M − H]⁻.
RT: retention time which corresponds to HPLC chromatogram of the crude extract in FIG. 1A.
MS: mass m/z
m/z represents [M − H]⁻.
RT: retention time which corresponds to HPLC chromatogram of the crude extract in FIG. 1A.
MS: mass spectrometry.
MF: molecular formula.
RA: relative abundance in percentage was calculated on purified fraction dry weight basis.
*indicates new compounds.

### Example 2—Characterisation

### Mass Spectrometry, Physiochemical Properties & Functional Groups

Tandem liquid chromatography-mass spectrometry (LC-MS/MS) was performed using a SCIEX TripleTOF 6600 (SCIEX, Framingham, MA, USA) mass spectrometer, using a Luna Omega capillary column (Phenomenex, C18 0.3×150 mm) at a flow rate of 7 μL/min. The gradient programme and buffers were the same as described for LC-MS above. Product ion (MS/MS) mass spectra were collected in negative ion mode with an accumulation time of 400 ins and a mass range of m/z 50 to 550. The source temperature was 250° C., the curtain gas was set to 35 psi, the ion source gas 1 and 2 were set to 25 and 30 psi, respectively, and the ion-spray voltage floating set to −4.5 kV. Data acquisition was performed with Analyst® TF 1.7.1 software (SCIEX, Framingham, MA, USA).

High-resolution mass spectrometry (HRMS) data were acquired using a Micromass Waters Xevo Q-ToF Ultima (quadrupole time-of-flight) mass spectrometer, in the negative ion mode, using MeOH as the solvent. The ionisation method was by electrospray ionisation (ESI).

Melting point data were acquired using a Gallenkamp MF-370 capillary tube melting point apparatus and are uncorrected. FTIR data were acquired on a Bruker Vertex 70 FTIR spectrometer using neat samples. Optical rotation data were acquired using a Jasco P-2000 polarimeter equipped with a 1 dm sample cell of volume 2 mL.

### Nuclear Magnetic Resonance Spectroscopy

Freeze-dried compounds were dissolved in CD₃OD. All NMR spectra were acquired on a Bruker 600 MHz AVANCE III NMR spectrometer (Bruker, Karlsruhe, Germany) equipped with a cryogenically cooled probe. ¹³C APT spectra were acquired for carbon chemical shifts. Two-dimensional ¹H-¹³C HSQC, ¹H-¹H COSY, ¹H-¹H TOCSY, ¹H-¹H HMBC, ¹H-¹H NOESY were collected at 290 K were used for structure elucidation. All spectra were recorded with a 1 s interscan delay using standard Bruker pulse sequences with an excitation sculpting scheme for solvent suppression. The structures of the compounds were confirmed by mapping the 1D and 2D NMR spectra as described in Wangchuk et al.

NMR data were analysed using Topspin v3.6.1 (Bruker, Billerica, MA, USA). The molecular structures and 2D NMR correlations were drawn using ChemDraw software (version 20.0.0.41).

Four known (compounds 1-3 and 5) and two novel (compounds 4 and 6) compounds were identified from the polar crude extract of U. metrosideros. The known compounds were identified here for the first time in the genus Uromyrtus.

### Compound Data

Compound 3: Brownish powder;

$${[ α ]}_{D}^{25} + 59.6$$

(c 0.08, MeOH); HRMS m/z 385.1868 [M-H]⁻ (calcd. 385.18679).

Compound 4: Brownish powder;

$${[ α ]}_{D}^{25} + 24.6$$

(c 0.06, MeOH); mp=188° C. (decomp); HRMS m/z 537.1600 [M-H]⁻ (calcd. 537.16137). IR (neat): 3209, 2966, 1696, 1607, 1536, 1443, 1317, 1223, 1095, 1032, 844, 800, 733 cm⁻¹.

Compound 5: Brownish powder; HRMS m/z 467.1916 [M-H]⁻ (calcd. 467.19227).

Compound 6: Brownish powder;

$${[ α ]}_{D}^{25} + 9.3$$

(c 0.08, MeOH); mp=176° C. (decomp); HRMS m/z 543.2443 [M-H]⁻ (calcd. 543.24470). IR (neat): 3340, 2926, 1692, 1610, 1535, 1446, 1319, 1209, 1072, 1035, 987, 932, 871, 801, 767, 742 cm⁻¹.

Detailed NMR characterisation of each compound 1-6 using the above equipment and techniques generated the data presented in Tables 2 & 3.

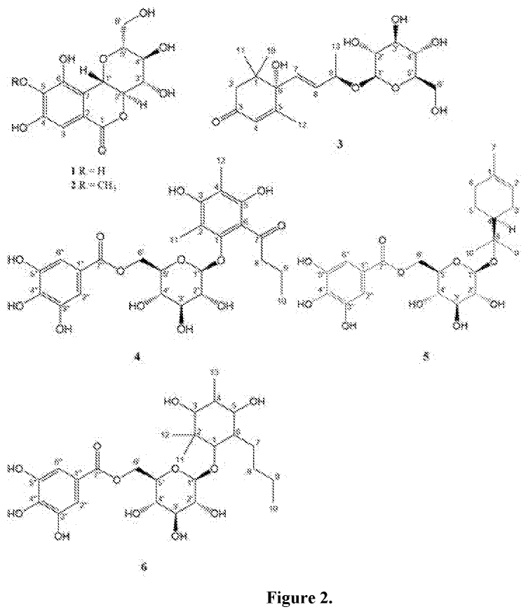

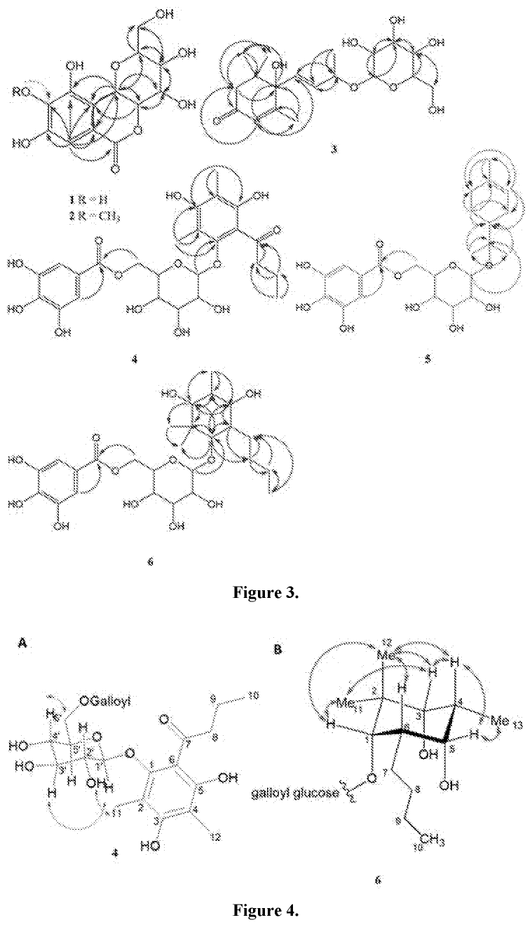

The chemical structures of compounds 1-6 and their HMBC correlations are given in FIGS. 2 and 3, respectively.

TABLE 2
¹H NMR spectroscopic data (δ) for compounds 1-6 isolated from Uromyrtus metrosideros.

| | | | | | | |
|---|---|---|---|---|---|---|
| No. position | 1 | 2 | 3 | 4 | 5 | 6 |

Aglycone

| | | | | | | | | | | | | |
|---|---|---|---|---|---|---|---|---|---|---|---|---|
| 1 | — | | — | | — | | — | | — | | 3.68 | s |
| 2 | — | | — | | 2.53 | d (17.1) | — | | 5.28 | d (4.5) | — | |
| | | | | | 2.15 | d (17.1) | | | | | | |
| 3 | 7.08 | s | 7.08 | s | — | | — | | 1.72 | m | 3.15 | d (2.5) |
| | | | | | | | | | 2.01 | m | | |
| 4 | — | | — | | 5.86 | quint (1.5) | — | | 1.69 | m | 1.87 | m |
| 5 | — | | — | | — | | | | 2.00 | m | 3.71 | brs, t |
| | | | | | | | | | 1.15 | m | | |
| 6 | — | | — | | — | | — | | 1.81 | dd (16.8, | 1.83 | m |
| | | | | | | | | | | 4.4) | | |
| | | | | | | | | | 1.90 | m | | |
| 7 | — | | — | | 5.85 | m | — | | 1.48 | s | 1.31 | m |
| | | | | | | | | | | | 1.39 | m |
| 8 | — | | 3.90 | s | 5.85 | m | 3.06 | m | — | | 1.65 | m |
| | | | | | | | | | | | 1.85 | m |
| 9 | | | | | 4.41 | m | 1.44 | m | 1.14 | s | 1.38 | m |
| 10 | | | | | 1.02 | s | 0.77 | t (7.6) | 1.18 | s | 0.95 | t (6.9) |
| 11 | | | | | 1.03 | s | 2.18 | s | | | 1.11 | s |
| 12 | | | | | 1.92 | d (1.5) | 2.00 | s | | | 0.83 | s |
| 13 | | | | | 1.28 | d (5.9) | | | | | 1.18 | d (6.9) |

Glucose

| | | | | | | | | | | | | |
|---|---|---|---|---|---|---|---|---|---|---|---|---|
| 1′ | 4.95 | d (10.5) | 4.96 | d (10.3) | 4.34 | d (7.9) | 4.49 | d (7.0) | 4.50 | d (7.3) | 4.37 | d (7.7) |
| 2′ | 3.67 | m | 3.68 | m | 3.17 | dd (8.9, 7.4) | 3.55 | dd (6.4, | 3.17 | dd (9.0, | 3.19 | m |
| | | | | | | | | 4.2) | | 7.9) | | |
| 3′ | 3.42 | t (9.1) | 3.43 | t (9.1) | 3.33 | t (8.6) | 3.44 | t (9.4) | 3.36 | t (8.9) | 3.38 | m |
| | | | | | | | | | | | | (overlapped) |
| 4′ | 3.80 | t (9.0) | 3.80 | t (9.0) | 3.24 | t (9.5) | 3.58 | t (6.9) | 3.32 | s | 3.38 | m |
| | | | | | | | | | | | | (overlapped) |
| 5′ | 4.03 | t (10.0) | 4.04 | t (10.0) | 3.23 | m | 3.33 | m | 3.54 | m | 3.49 | m |
| 6′ | 4.02 | d (9.6) | 4.02 | d (10.1) | 3.84 | dd (11.8, | 4.35 | dd (11.8, | 4.46 | dd (11.6, | 4.54 | dd (11.6, 2.5) |
| | | | | | | 2.0) | | 2.1) | | 1.8) | | |
| | 3.67 | d | 3.68 | d (8.8) | 3.63 | dd (11.8, | 4.27 | dd (11.8, | 4.32 | dd (11.9, | 4.44 | dd (11.6, 5.8) |
| | | (overlapped) | | | | 5.6) | | 4.8) | | 6.8) | | |

Galloyl

| | | | | | | |
|---|---|---|---|---|---|---|
| 1″ | — | | — | | — | |
| 2″, 6″ | 7.02 | s | 7.05 | s | 7.08 | s |
| 3″, 5″ | — | | — | | — | |
| 4″ | — | | — | | — | |
| 7″ | — | | — | | — | |

¹H NMR data (δ in ppm, J in Hz) were recorded at 600 MHz using in CD₃OD.

TABLE 3
¹³C NMR spectroscopic data (δ) for compounds 1-6 isolated from Uromyrtus metrosideros.

| | | | | | | |
|---|---|---|---|---|---|---|
| No. position | 1 | 2 | 3 | 4 | 5 | 6 |

Aglycone

| | | | | | | |
|---|---|---|---|---|---|---|
| 1 | 166.5 | 165.8 | 42.5 | 154.8 | 135.1 | 85.9 |
| 2 | 114.2 | 119.5 | 50.8 | 111.2 | 121.8 | 40.8 |
| 3 | 111.1 | 111.1 | 201.2 | 161.2 | 28.3 | 81.7 |
| 4 | 147.3 | 152.4 | 127.3 | 109.2 | 45.2 | 37.4 |
| 5 | 141.2 | 142.4 | 167.5 | 160.4 | 25.2 | 74.7 |
| 6 | 143.6 | 149.5 | 80.0 | 112.6 | 32.1 | 43.0 |
| 7 | 117.2 | 117.4 | 131.6 | 209.4 | 23.7 | 30.6 |
| 8 | | 60.9 | 135.4 | 47.0 | 81.5 | 30.1 |
| 9 | | | 77.4 | 19.4 | 23.1 | 24.0 |
| 10 | | | 24.8 | 14.2 | 25.4 | 14.6 |
| 11 | | | 23.5 | 9.6 | | 25.8 |
| 12 | | | 19.7 | 8.1 | | 26.3 |
| 13 | | | 21.3 | | | 15.6 |

Glucose

| | | | | | | |
|---|---|---|---|---|---|---|
| | 74.3 | 74.2 | 102.8 | 105.5 | 98.7 | 104.1 |
| 2 | 83.0 | 83.1 | 75.3 | 75.7 | 75.3 | 75.9 |
| 3′ | 71.9 | 71.9 | 78.1 | 77.5 | 72.3 | 77.9 |
| 4 | 75.8 | 75.7 | 71.7 | 71.1 | 78.4 | 72.3 |
| 5 | 81.5 | 81.5 | 78.1 | 75.4 | 75.6 | 75.6 |
| 6 | 62.8 | 62.7 | 62.9 | 63.6 | 65.6 | 64.7 |

Gallic acid

| | | | |
|---|---|---|---|
| 1″ | 121.4 | 121.6 | 121.6 |
| 2″, 6″ | 110.2 | 110.3 | 110.3 |
| 3″, 5″ | 146.5 | 146.6 | 146.6 |
| | 140.1 | 139.9 | 139.9 |
| 7″ | 168.5 | 168.4 | 168.3 |

¹³C NMR data (δ in ppm) recorded at 150 MHz using CD₃OD.

Compounds 1 and 2 were closely related and were identified as norbergenin (m/z 313) and bergenin (m/z 327), respectively. Norbergenin (compound 1) is the 0-demethylated derivative of bergenin (compound 2), which belongs to the chemical class of trihydroxybenzoic acid glycoside. Both compounds showed ¹H and ¹³C NMR chemical shifts, ¹H multiplicities, and 2D NMR correlations (FIG. 3, Tables 2 and 3) identical to those previously reported (Saijo, et al.; Jouwa, et al.). Bergenin (compound 2) has been identified in plant species as reviewed by Bajracharya et al. and Rastogi et al. Among Myrtaceae, the occurrence of both bergenin (compound 2) and norbergenin (compound 1) has been reported only in Syzygium species (Kopanski, et al.; Qiu, et al.; Sheng-Yuan, et al.). This is the first time these two compounds 1 and 2 have been reported in the genus Uromyrtus.

Compound 3 was identified as roseoside (m/z 385), which belongs to the chemical class of fatty acyl glycosides of mono- and disaccharides. The ¹H and ¹³C NMR chemical shifts, ¹H NMR multiplicities and 2D NMR correlations were identical to those of roseoside previously reported from other plant species (Yamano et al.; Mamadalieva, et al.). The aglycone methylene proton chemical shifts of 3 (H-2, 2.53 and 2.15 ppm, J=17.1 Hz) were consistent with the (6S,9R)-roseoside stereoisomer specifically, which was further supported by the sign of its specific rotation. This is the first time that compound 3 has been reported in a plant of the genus Uromyrtus.

Compounds 4-6 belong to the chemical class of galloyl glucosides. All contained galloyl moieties characterised by a symmetric aromatic proton singlet (H-2″ and 1H-6″, 2H) at 7.02-7.08 ppm, which correlated to five characteristic galloyl moiety ¹³C NMR signals (121.4, 110.2, 146.5, 140.1, 168.5 ppm; Tables 2-3, FIG. 3) in the HMBC spectrum. The galloyl moieties were each linked to a glucose unit via a single bond through C-6′ (at 63.6-65.6 ppm) attached to two protons (doublet of doublets) detected at 4.2-4.4 ppm (J=12 and 5-7, 1H) and 4.3-4.6 ppm (J=12 and 2, 1H) which showed HMBC correlations to C-7″ of the galloyl moiety. The aglycones of compounds 4-6 were all linked to glucose via C-1′-O, which was readily identified from its more de-shielded chemical shift (99-105 ppm).

Compound 4 was isolated as an amorphous solid compound and exhibited a specific rotation of

$${[ α ]}_{D}^{25} + 24.9$$

(c 0.06, MeOH) and an [M-H]⁻ ion peak at m/z 537.1600 (calcd. 537.16137, −2.54 ppm) consistent with the molecular formula C₂₅H₃₀O₁₃. The galloyl glucoside moiety in compound 4 was linked to a β-D-glucopyranosyl unit which had the same chemical shifts (Tables 2 and 3) and HMBC correlations (FIG. 3) to those reported for lawsoniaside A

$${[ α ]}_{D}^{25} + 1.5$$

(c 0.50, MeOH)](Cuong, et al.), which lacks the galloyl moiety found in compound 4. The aglycone showed two methyl singlets at 2.18 and 2.00 ppm (Table 2) coupled to quaternary carbons (C-2 and C-4) (Table 3) at chemical shifts characteristic of aromatic carbons (at 111.2 and 109.2 ppm), supporting the asymmetry of this moiety. Two other aromatic carbons (C-3 and C-5) were more downfield (at 161.2 and 160.4 ppm, respectively) as they were substituted with OH groups. The CH₃—CH₂—CH₂—C(O)— moiety was supported from the ¹H NMR multiplicities and their chemical shifts (0.77 (t), 1.44 (m), 3.06 (m), as well as their ¹³C NMR chemical shifts of C-10, C-9, C-8 and C-7, at 14.2, 19.4, 47.0 and 209.4 ppm (Tables 2 and 3), respectively and further supported by COSY and TOCSY correlations. The relative stereochemistry of aglycone was determined using NOESY correlations (FIG. 4A). This new compound has been named galloyl-lawsoniaside A.

Compound 5 was identified as α-terpineol 8-O-β-D-(6-O-galloyl)glucopyranoside. It exhibited an [M-H]⁻ ion peak at m/z 467.1916 (calcd. 467.19227, −1.44 ppm) consistent with the molecular formula C₂₃H₃₂O₁₀. Compound 5 showed ¹H and ¹³C NMR chemical shifts and 2D NMR correlations in overall agreement with those previously reported (Kikuzaki, et al.). The 4R and 4S stereoisomers can be distinguished in CD₃OD by their relative differences in the chemical shifts of C-9 and C-10. Based on this, compound 5 can be assigned as (4S)-α-terpineol 8-O-β-D-(6-O-galloyl)glucopyranoside. α-Terpineol 8-O-β-D-(6-O-galloyl)glucopyranoside has been reported only in one plant species, from the berries of Pimenta dioica (Myrtaceae) (Kikuzaki, et al.). This is the first time that compound 4 has been reported in the genus Uromyrtus.

Compound 6 was isolated as an amorphous solid compound and exhibited a specific rotation of

$${[ α ]}_{D}^{25} + 9.3$$

(c 0.08, MeOH) and displayed an [M-H]⁻ ion peak at m/z 543.2442 (calcd. 543.24470, −0.74 ppm) consistent with the molecular formula C₂₆H₄₀O₁₂. Because all carbons of the aglycone were coupled to at least one proton (except for C-2), the structure elucidation of this moiety was resolved by COSY, TOCSY correlations and verified by HMBC correlations (FIG. 3). The structure of the aglycone was similar to that of compound 4, as it had two OH moieties attached to C-3 and C-5, and a butyl moiety at C-6 (Tables 2 and 3, FIG. 2). The structure of CH₃—CH₂—CH₂—CH₂— moiety was supported by the ¹H NMR multiplicities and their chemical shifts (H-10 0.95 (t), H-9 1.38 (m), H-8 1.85 (m) and 1.65, H-7 1.39 (m) and 1.31 (m)), as well as chemical shifts of C-10, C-9, C-8 and C-7, at 14.6, 24.0, 30.1 and 30.6 ppm (Tables 2 and 3). Two methyl groups were coupled to the quaternary carbon C-2, characterised by two ¹H NMR singlets (3H each) at 1.11 and 0.83 ppm, and their respective carbon chemical shifts at 25.8 and 26.3 ppm. The remaining methyl moiety was coupled to C-4 as a doublet at 1.18 ppm and its respective carbon chemical shift at 15.6 ppm. The relative stereochemistry (FIG. 4B) of aglycone was determined using ROESY correlations. The cyclohexane ring consisted of three equatorial protons H-1, H-3 and H-5 and two axial protons H-4 and H-6. Both H-1 and H-3 were correlated to CH₃-11 and CH₃-12; H-5 was correlated to H-4 and CH₃-13; H-4 was correlated to CH₃-12 and CH₃-12 was not correlated to CH₃-13. Finally, H-6 was correlated to CH₃-12, which was not correlated to H-7.

### Example 3—Cytotoxicity Assessment

### Sample Preparation

1 mg/ml stock solutions of compounds 3-6 and PCE were prepared by initially dissolving 1 mg weighed compounds in 10 μL of DMSO and subsequently diluting with 990 μL of RPMI-1640 media without L-glutamine (Gibco Thermo Fisher Scientific, Waltham, MA, USA).

PBMCs from four healthy donors were isolated from buffy coats using Lymphoprep™ density gradient medium (STEMCELL Technologies, Vancouver, Canada), according to the manufacturer's instructions. Isolated cells were cryopreserved in 90% heat-inactivated foetal calf serum (FCS) (Bovogen Biologicals) with 10% dimethyl sulfoxide (DMSO; Sigma-Aldrich, St. Louis, MO, USA) using vapour phase nitrogen until required. On the day of the experiment, PBMCs were thawed and removed from the freezing medium by washing with 9 mL pre-warmed culture medium and centrifugation at 300×g for 5 min. An R10 culture medium of RPMI-1640 media without L-glutamine (Gibco Thermo Fisher Scientific, Waltham, MA, USA), supplemented with 10% heat-inactivated foetal bovine serum (FBS; Bovogen Biologicals, Christchurch, New Zealand), 10,000 units/mL of penicillin+10,000 μg/mL of streptomycin (Thermo Fisher Scientific), and 1× GlutaMAX (Thermo Fisher Scientific) was used for all experiments. The cells were then treated with 10 μg/mL DNase solution I (STEMCELL Technologies) in 5 mL R10 for 1 h at 37° C. with 5% CO₂ to reduce clumping. After incubation, the enzyme was removed by two additional washes with R10. Thawed PBMCs were counted using a CASY cell counter (Roche Innovatis, Germany), and cell densities were adjusted to seed 100,000 cells/well into 96-well plates in a final volume of 100 μL culture medium.

### Cytotoxicity Screening

PBMC viability was monitored in vitro following exposure to compounds 3-6 and PCE using the RealTime-Glo™ MT cell viability assay. All experiments were performed according to the manufacturer's instructions. For all assays, cells were cultured in 96-well, TC-treated, clear flat-bottom, white polystyrene plates (Corning®). Initially, several concentrations of the purified compounds (100, 50, and 10 μg/mL) were assessed in quadruplicates using 10⁶ cells/ml from a single donor over 24 h. An optimal concentration of 10 μg/mL was determined. Next, PBMCs of three donors were treated in six replicates with 10 μg/mL of U. metrosideros compounds or PCE, 4% lysis buffer, or remained untreated. Treatment-matched vehicle wells were included to control for inherent compound colour and background luminescence. All culture plates were kept in the dark at 37° C., with 5.5% CO₂ for 24 h. Luminescence (RLU) was measured during treatment at 1, 6, and 24 h using a FLUO®star Omega Microplate Reader (BMG LABTECH). Finally, the background values were subtracted, and PBMC viability was determined, referencing the untreated samples as 100% viability/pro-substrate metabolism.

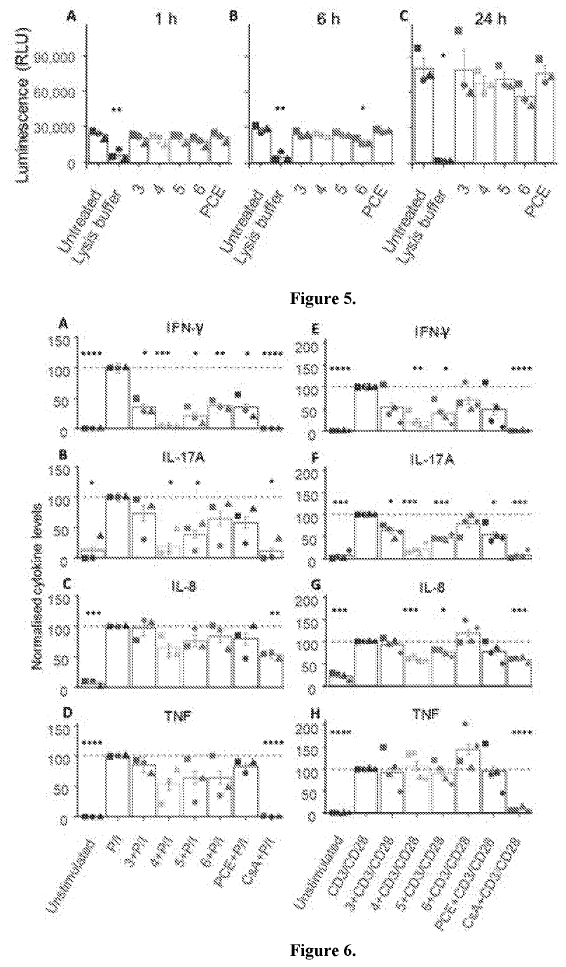

The luminescent signals (relative luminescent units; RLU), produced by the cells treated with 10 μg/mL (compounds 3-5) and PCE, did not significantly differ from the untreated control cells at 1, 6, and 24 h post-treatment (FIG. 5), thus indicating no change in cell metabolic activity (metabolism of the reagent pro-substrate or surrogate viability) up to 24 h. A slight but significant reduction in RLU was detected in the cells treated with compound 6 at 6 h post-treatment, representing a 37.8% decrease in metabolic activity compared to the untreated control. However, the observed difference of 30.6% in these samples was not statistically significant by 24 h. Conversely, as expected, lysis buffer treatment caused a rapid reduction of cell viability, as evidenced by significantly reduced luminescent signals of 74.8%, 84.5%, and 99.1% at 1, 6, and 24 h, respectively.

After 20 h of treatment, the release of four cytokines—IFN-γ, IL-17A, IL-8, and TNF were measured in the cell culture supernatant (FIG. 6A-D).

Cytotoxicity data were compared using T test at p<0.05 with untreated and stimulated groups as a reference, respectively, and plotted using ‘rstatix’, ‘dplyr’ and ‘ggpubr’ RStudio packages.

These data demonstrated that 10 μg/mL (compounds 3-6) and PCE were not toxic to human PBMCs for up to 24 h. Accordingly, these conditions were selected for the subsequent immunoassays.

### Example 4—Anti-Inflammatory Activity

### Human Cytokine Suppression Assays

For our cytokine suppression assays, inflammatory cytokines (IFN-γ, IL-17A, IL-8, and TNF) were induced in two ways. First, the PBMCs from three donors were activated with a cell stimulation cocktail of 50 ng/mL phorbol 12-myristate 13-acetate and 1 μg/mL ionomycin (P/I; eBioscience). P/1-stimulated cells were treated with 10 μg/mL of U. metrosideros compounds, PCE, CsA (Sigma-Aldrich), or remained untreated. Second, the PBMCs from four donors were cultured in the presence of Dynabeads Human T-Activator CD3/CD28 (Gibco) at a cell-to-bead ratio of 1:1. Anti-CD3/anti-CD28-stimulated PBMC were treated with 10 μg/mL of U. metrosiceros compounds, PCE, CsA, or remained untreated. The cell culture plates were incubated overnight at 37° C., 5.5% CO₂ for all screening experiments. After incubation, the samples were centrifuged at 500×g for 5 min, and the culture supernatants were collected and stored at −80° C. until cytokine analysis. On the day of the experiment, cytokine concentrations were determined using a standard LEGENDplex™ human inflammation panel 1 (BioLegend®), LSRFortessa (BD), and LEGENDplex™ software (version 2022-02-10, Qognit, Inc.).

For the initial screening, after 20 h of treatment, the release of four cytokines—IFN-γ, IL-17A, IL-8, and TNF were measured in the cell culture supernatant (FIG. 6A-D). The data revealed that all treatments significantly suppressed IFN-γ secretion. Compound 4 was the most effective in suppressing its release (95.3%), followed by compounds 5, PCE, 3, and 6 (79.6, 64.8, 64.4, 62.3%, respectively) (FIG. 6A). Compounds 4 and 5 also significantly reduced IL-17A levels by 79.1 and 60.9%, respectively (FIG. 6B). Promisingly, compound 4-induced inhibition of IFN-γ and IL-17A was similar to CsA results (99.9% and 88.9% decrease).

For the secondary screening, after 20 h, IFN-γ, IL-17A, IL-8, and TNF release was measured in the cell culture supernatant (FIG. 6E-H). Across donors, compounds 4 and 5 strongly suppressed Dynabead-induced IFN-γ and IL-17A secretion (78.5 and 59.3%, and 80.9 and 53.7%, respectively) and moderately inhibited IL-8 release by 40.5, and 24.3% (FIG. 6G). This inhibition was similar to that observed by CsA treatment (99.2, 92.9, and 40.7% for IFN-γ, IL-17A, and IL-8, respectively). Compound 3 and PCE moderately reduced IL-17A release by 38.2 and 45.8%, respectively (FIG. 6F).

Cytokine release data were compared using T test at p<0.05 with untreated and stimulated groups as a reference, respectively, and plotted using ‘rstatix’, ‘dplyr’ and ‘ggpubr’ RStudio packages.

### REFERENCES

Bajracharya G B. Diversity, pharmacology and synthesis of bergenin and its derivatives: Potential materials for therapeutic usages. Fitoterapia 2015; 101: 133-52.
Cuong N X, Nhiem N X, Thao N P, Nam N H, Dat N T, Anh H L T, et al. Inhibitors of osteoclastogenesis from Lawsonia inermis leaves. Bioorganic & Medicinal Chemistry Letters 2010; 20: 4782-4.
Jouwa Tameye N S, Mvot Akak C, Mouthé Happi G, Frese M, Stammler H-G, Neumann B. et al. Antioxidant norbergenin derivatives from the leaves of Diospyros gilletii De Wild (Ebenaceae). Phytochemistry Letters 2020; 36: 63-7.
Kikuzaki H, Sato A, Mayahara Y, Nakatani N. Galloylglucosides from berries of Pimenta dioica. Journal of Natural Products 2000; 63: 749-52.
Kopanski L. Schnelle G. Isolation of bergenin from barks of Syzygium cumini. Planta medica 1988; 54: 572-.
Mamadalieva N, Sharopov F, Girault J-IP, Wink M, Lafont R. Phytochemical analysis and bioactivity of the aerial parts of Abutilon theophrasti (Malvaceae), a medicinal weed. Natural Product Research 2014; 28: 1777-9.
Qiu J. Chen X. Liang P, Zhang L, Xu Y, Gong M, et al. Integrating approach to discover novel bergenin derivatives and phenolics with antioxidant and anti-inflammatory activities from bio-active fraction of Syzygium brachythyrsum. Arabian Journal of Chemistry 2021: 103507.
Rastogi S, Rawat A K S. A comprehensive review on bergenin, a potential hepatoprotective and antioxidative phytoconstituent. Herba Polonica 2008; 54: 66-79.
Saijo R, Nonaka G-i, Nishioka I. Gallic acid esters of bergenin and norbergenin from Mallotus japonicus. Phytochemistry 1990; 29: 267-70.
Sheng-Yuan Z, Yuan-Bei Z, Da-Du L, Ming Z, Jun-Fang L, Zheng-Hui W, et al. Chemical constituents from α-glucosidase inhibitory active part in stems and leaves of Syzygium jambos. Natural Product Research And Development 2018; 30: 1950-6.
Wangchuk P. Navarro S. Shepherd C, Keller P A, Pyne S G, Loukas A. Diterpenoid alkaloids of Aconitum laciniatum and mitigation of inflammation by 14-O-acetylneoline in a murine model of ulcerative colitis. Scientific Reports 2015; 5: 1-10.
Yamano Y, Ito M. Synthesis of optically active vomifoliol and roseoside stereoisomers. Chemical and Pharmaceutical Bulletin 2005; 53: 541-6.
Ziaullah, H. P. V. R., Sonochemical enzyme-catalyzed regioselective acylation of flavonoid glycosides. Bioorganic Chemistry, 2016, 65 17-25.
Zhao, Y. et al. Isolation, synthesis and anti-hepatitis B virus evaluation of p-hydroxyacetophenone derivatives from Artemisia capillaris, Bioorganic & Medicinal Chemstry Letters, 2015, 25, 1509-1514.
Zhu, X. et al., Identification of a new benzophenone from Psidium guajava L. leaves and its antineoplastic effects on human colon cancer cells, Food & Function, 2019, 10, 4189.

1. A compound of formula (I): 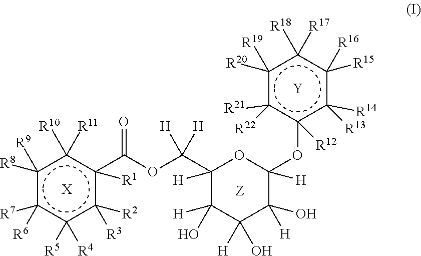
or a pharmaceutically acceptable salt thereof, wherein:
X is cyclohexane, cyclohexene, cyclohexadiene, or benzene, wherein;
when X is benzene R¹, R³, R⁵, R⁷, R⁹ and R¹¹ are absent;
when X is cyclohexene, either: R¹ and R³ are absent and R¹C—CR²R³ is taken to be C═CR²; R³ and R⁵ are absent and R²R³C—CR⁴R⁵ is taken to be R²C═CR⁴; R⁵ and R⁷ are absent and R⁴R⁵C—CR⁶R⁷ is taken to be R⁴C═CR⁶; R⁷ and R⁹ are absent and R⁶R⁷C—CR⁸R⁹ is taken to be R⁶C═CR⁸; R⁹ and R¹¹ are absent and R⁸R⁹C—CR¹⁰R¹¹ is taken to be R⁸C═CR¹⁰; or R¹¹ and R¹ are absent and R¹⁰R¹¹C—CR¹ is taken to be R¹⁰C═C;
when X is cyclohexadiene, either: R¹, R³, R⁵ and R⁷ are absent and R¹C—CR²R³ and R⁴R⁵C—CR⁶R⁷ are taken to be C═CR² and R⁴C═CR⁶; R¹, R³, R⁷ and R⁹ are absent and R¹C—CR²R³ and R⁶R⁷C—CR⁸R⁹ are taken to be C═CR² and R⁶C═CR⁸; R¹, R³, R⁹ and R¹¹ are absent and R¹C—CR²R³ and R⁸R⁹C—CR¹⁰R¹¹ are taken to be C═CR² and R⁸C═CR¹⁰; R³, R⁵, R⁷ and R⁹ are absent and R²R³C—CR⁴R⁵ and R⁶R⁷C—CR⁸R⁹ are taken to be R²C═CR⁴ and R⁶C═CR⁸; R³, R⁵, R⁹ and R¹¹ are absent and R²R³C—CR⁴R⁵ and R⁸R⁹C—CR¹⁰R¹¹ are taken to be R²C═CR⁴ and R⁸C═CR¹⁰; R³, R⁵, R¹¹ and R¹ are absent and R²R³C—CR⁴R⁵ and R¹⁰R¹¹C—CR¹ are taken to be R²C═CR⁴ and R¹⁰C═C; R⁵, R⁷, R⁹ and R¹¹ are absent and R⁴R⁵C—CR⁶R⁷ and R⁸R⁹C—CR¹⁰R¹¹ are taken to be R⁴C═CR⁶ and R⁸C═CR¹⁰; R⁵, R⁷, R¹¹ and R¹ are absent and R⁴R⁵C—CR⁶R⁷ and R¹⁰R¹¹C—CR¹ are taken to be R⁴C═CR⁶ and R¹⁰C═C; or R⁷, R⁹, R¹¹ and R¹ are absent and R⁶R⁷C—CR⁸R⁹ and R¹⁰R¹¹C—CR¹ are taken to be R⁶C═CR⁸ and R¹⁰C═C;
Y is cyclohexane, cyclohexene, cyclohexadiene, or benzene, wherein;
when Y is benzene R¹², R¹⁴, R¹⁶, R¹⁸, R²⁰ and R²² are absent;
when Y is cyclohexene, either: R¹² and R¹⁴ are absent and R¹²C—CR¹³R¹⁴ is taken to be C═CR¹³; R¹⁴ and R¹⁶ are absent and R¹³R¹⁴C—CR¹⁵R¹⁶ is taken to be R¹³C═CR¹⁵; R¹⁶ and R¹⁸ are absent and R¹⁵R¹⁶C—CR¹⁷R¹⁸ is taken to be R¹⁵C═CR¹⁷; R¹⁸ and R²⁰ are absent and R¹⁷R¹⁸C—CR¹⁹R²⁰ is taken to be R¹⁷C═CR¹⁹; R²⁰ and R²² are absent and R¹⁹R²⁰C—CR²¹R²² is taken to be R¹⁹C═CR²¹; or R²² and R¹² are absent and R²¹R²²C—CR¹² is taken to be R²¹C═C;
when Y is cyclohexadiene, either: R¹², R¹⁴, R¹⁶ and R¹⁸ are absent and R¹²C—CR¹³R¹⁴ and R¹⁵R¹⁶C—CR¹⁷R¹⁸ are taken to be C═CR¹³ and R¹⁵C═CR¹⁷; R¹², R¹⁴, R¹⁸ and R²⁰ are absent and R¹²C—CR¹³R¹⁴ and R¹⁷R¹⁸C—CR¹⁹R²⁰ are taken to be C═CR¹³ and R¹⁷C═CR¹⁹; R¹², R¹⁴, R²⁰ and R²² are absent and R¹²C—CR¹³R¹⁴ and R¹⁹R²⁰C—CR²¹R²² are taken to be C═CR¹³ and R¹⁹C═CR²¹; R¹⁴, R¹⁶, R¹⁸ and R²⁰ are absent and R¹³R¹⁴C—CR¹⁵R¹⁶ and R¹⁷R¹⁸C—CR¹⁹R²⁰ are taken to be R¹³C═CR¹⁵ and R¹⁷C═CR¹⁹; R¹⁴, R¹⁶, R²⁰ and R²² are absent and R¹³R¹⁴C—CR¹⁵R¹⁶ and R¹⁹R²⁰C—CR²¹R²² are taken to be R¹³C═CR¹⁵ and R¹⁹C═CR²¹; R¹⁴, R¹⁶, R²² and R¹² are absent and R¹³R¹⁴C—CR¹⁵R¹⁶ and R²¹R²²C—CR¹² are taken to be R¹³C═CR¹⁵ and R²¹C═C; R¹⁶, R¹⁸, R²⁰ and R²² are absent and R¹⁵R¹⁶C—CR¹⁷R¹⁸ and R¹⁹R²⁰C—CR²¹R²² are taken to be R¹⁵C═CR¹⁷ and R¹⁹C═CR²¹; R¹⁶, R¹⁸, R²² and R¹² are absent and R¹⁵R¹⁶C—CR¹⁷R¹⁸ and R²¹R²²C—CR¹² are taken to be R¹⁵C═CR¹⁷ and R²¹C═C; or R¹⁸, R²⁰, R²² and R¹² are absent and R¹⁷R¹⁸C—CR¹⁹R²⁰ and R²¹R²²C—CR¹² are taken to be R¹⁷C═CR¹⁹ and R²¹C═C;
at least one of R¹² to R²² is —C₁-C₁₀ alkyl;
each of R¹ to R²² when present is otherwise independently selected from the group consisting of —H, —OH, halogen, —CN, —SH, —C₁-C₁₀ alkyl, —C₂-C₁₀ alkenyl, —C₂-C₁₀ alkynyl, —C(O)C₁-C₁₀ alkyl, —C(O)C₂-C₁₀ alkenyl, —C(O)C₂-C₁₀ alkynyl, —OC₁-C₁₀ alkyl, —OC₂-C₁₀ alkenyl, —OC₂-C₁₀ alkynyl, —OC(O)C₁-C₁₀ alkyl, —OC(O)C₂-C₁₀ alkenyl, —OC(O)C₂-C₁₀ alkynyl, —OC(O)NHC₁-C₁₀ alkyl, —OC(O)NHC₂-C₁₀ alkenyl, —OC(O)NHC₂-C₁₀ alkynyl, and —N(R²³)₂, or one or more of R¹³ and R¹⁴, R¹⁵ and R¹⁶, R¹⁷ and R¹⁸, R¹⁹ and R²⁰, and R²¹ and R²², taken together, are ═O, ═S, or ═NR²³ except when Y is cyclohexane in which case not both of R¹⁵ and R¹⁶, and R¹⁹ and R²⁰, taken together, are ═O; and
each R²³ is independently selected from the group consisting of H and —C₁-C₁₀ alkyl.

2. The compound or pharmaceutically acceptable salt according to claim 1, wherein X is benzene.

3. The compound or pharmaceutically acceptable salt according to claim 2, wherein each of R², R⁴, R⁶, R⁸ and R¹⁰ are independently selected from the group consisting of —H and —OH.

4. (canceled)

5. The compound or pharmaceutically acceptable salt according to claim 1, wherein Y is cyclohexane, cyclohexene, or benzene.

6. The compound or pharmaceutically acceptable salt according to claim 5, wherein each of R¹² to R²² when present, apart from the one that is —C₁-C₁₀ alkyl, are independently selected from the group consisting of —H, —OH, —C₁-C₁₀ alkyl, —C(O)C₁-C₁₀ alkyl, or one or more of R¹³ and R¹⁴, R¹⁵ and R¹⁶, R¹⁷ and R¹⁸, R¹⁹ and R²⁰, and R²¹ and R²², taken together, are ═O, subject to the proviso that when Y is cyclohexane not both of R¹⁵ and R¹⁶, and R¹⁹ and R²⁰, taken together, are ═O.

7. The compound or pharmaceutically acceptable salt according to claim 6, wherein:
Y is benzene, R¹³ and R²¹ are independently selected from the group consisting of —C(O)C₁-C₁₀ alkyl and —C₁-C₁₀ alkyl, R¹⁵ and R¹⁹ are —OH, and R₁₇ is —C₁-C₁₀ alkyl;
Y is cyclohexane, R¹³, R¹⁴, R¹⁷, R¹⁸, R²¹ and R²² are independently selected from the group consisting of —H and —C₁-C₁₀ alkyl, at least one of which being —C₁-C₁₀ alkyl, and R¹², R¹⁵, R¹⁶, R¹⁹ and R²⁰ are independently selected from the group consisting of —H and —OH;
Y is cyclohexene, R¹⁴ and R¹⁶ are absent and R¹³R¹⁴C—CR¹⁵R¹⁶ is taken to be R¹³C═CR¹⁵, or R²⁰ and R²² are absent and R¹⁹R²⁰C—CR²¹R²² is taken to be R¹⁹C═CR²¹, R¹⁷ and R¹⁸ taken together are ═O, R¹² is selected from the group consisting of —H and —OH, at least one of R¹³, R¹⁴, R²¹ and R²² is —C₁-C₁₀ alkyl, and the remainder of R¹² to R²² are —H; or
Y is cyclohexene, R¹⁶ and R¹⁸ are absent and R¹⁵R¹⁶C—CR¹⁷R¹⁸ is taken to be R¹⁵C═CR¹⁷, R¹⁷ is —C₁-C₁₀ alkyl, and the remainder of R¹² to R²² are —H.

8. (canceled)

9. (canceled)

10. (canceled)

11. The compound or pharmaceutically acceptable salt according to claim 1, wherein Z represents α- or β-, D- or L-, glucopyranose, mannopyranose or galactopyranose.

12. (canceled)

13. The compound or pharmaceutically acceptable salt according to claim 1, wherein the compound is selected from the group consisting of:

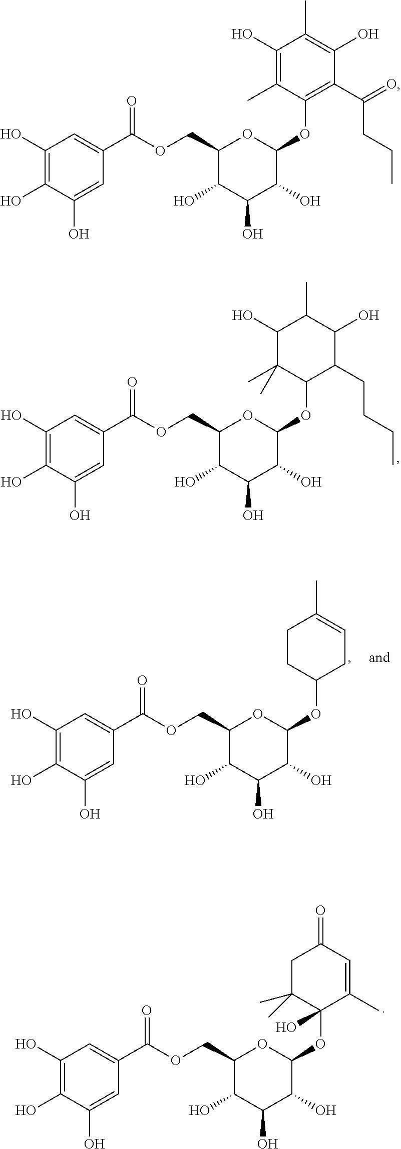 [SMILES: CCCC(=O)c1c(O)c(C)c(O)c(C)c1O[C@@H]1O[C@H](COC(=O)c2cc(O)c(O)c(O)c2)[C@@H](O)[C@H](O)[C@H]1O ; CCCCC1C(O)C(C)C(O)C(C)(C)C1O[C@@H]1O[C@H](COC(=O)c2cc(O)c(O)c(O)c2)[C@@H](O)[C@H](O)[C@H]1O ; CC1=CCC(O[C@@H]2O[C@H](COC(=O)c3cc(O)c(O)c(O)c3)[C@@H](O)[C@H](O)[C@H]2O)CC1 ; CC1=CC(=O)CC(C)(C)[C@@]1(O)O[C@@H]1O[C@H](COC(=O)c2cc(O)c(O)c(O)c2)[C@@H](O)[C@H](O)[C@H]1O]

14. (canceled)

15. A method for preventing or treating an inflammatory disease or disorder in a subject, comprising administering to the subject an effective amount of a compound of formula (II): 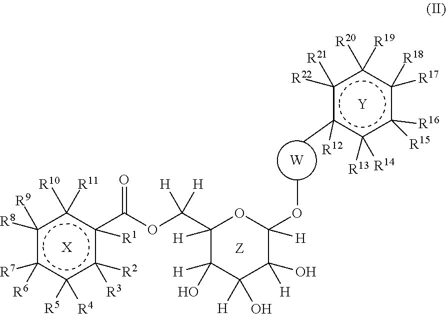
or a pharmaceutically acceptable salt thereof, wherein:
X is cyclohexane, cyclohexene, cyclohexadiene, or benzene, wherein;
when X is benzene R¹, R³, R⁵, R⁷, R⁹ and R¹¹ are absent;
when X is cyclohexene, either: R¹ and R³ are absent and R¹C—CR²R³ is taken to be C═CR²; R³ and R⁵ are absent and R²R³C—CR⁴R⁵ is taken to be R²C═CR⁴; R⁵ and R⁷ are absent and R⁴R⁵C—CR⁶R⁷ is taken to be R⁴C═CR⁶; R⁷ and R⁹ are absent and R⁶R⁷C—CR⁸R⁹ is taken to be R⁶C═CR⁸; R⁹ and R¹¹ are absent and R⁸R⁹C—CR¹⁰R¹¹ is taken to be R⁸C═CR¹⁰; or R¹¹ and R¹ are absent and R¹⁰R¹¹C—CR¹ is taken to be R¹⁰C═C;
when X is cyclohexadiene, either: R¹, R³, R⁵ and R⁷ are absent and R¹C—CR²R³ and R⁴R⁵C—CR⁶R⁷ are taken to be C═CR² and R⁴C═CR⁶; R¹, R³, R⁷ and R⁹ are absent and R¹C—CR²R³ and R⁶R⁷C—CR⁸R⁹ are taken to be C═CR² and R⁶C═CR⁸; R¹, R³, R⁹ and R¹¹ are absent and R¹C—CR²R³ and R⁸R⁹C—CR¹⁰R¹¹ are taken to be C═CR² and R⁸C═CR¹⁰; R³, R⁵, R⁷ and R⁹ are absent and R²R³C—CR⁴R⁵ and R⁶R⁷C—CR⁸R⁹ are taken to be R²C═CR⁴ and R⁶C═CR⁸; R³, R⁵, R⁹ and R¹¹ are absent and R²R³C—CR⁴R⁵ and R⁸R⁹C—CR¹⁰R¹¹ are taken to be R²C═CR⁴ and R⁸C═CR¹⁰; R³, R⁵, R¹¹ and R¹ are absent and R²R³C—CR⁴R⁵ and R¹⁰R¹¹C—CR¹ are taken to be R²C═CR⁴ and R¹⁰C═C; R⁵, R⁷, R⁹ and R¹¹ are absent and R⁴R⁵C—CR⁶R⁷ and R⁸R⁹C—CR¹⁰R¹¹ are taken to be R⁴C═CR⁶ and R⁸C═CR¹⁰; R⁵, R⁷, R¹¹ and R¹ are absent and R⁴R⁵C—CR⁶R⁸ and R¹⁰R¹¹C—CR¹ are taken to be R⁴C═CR⁶ and R¹⁰C═C; or R⁷, R⁹, R¹¹ and R¹ are absent and R⁶R⁷C—CR⁸R⁹ and R¹⁰R¹¹C—CR¹ are taken to be R⁶C═CR⁸ and R¹⁰C═C;
Y is cyclohexane, cyclohexene, cyclohexadiene, or benzene, wherein;
when Y is benzene R¹², R¹⁴, R¹⁶, R¹⁸, R²⁰ and R²² are absent;
when Y is cyclohexene, either: R¹² and R¹⁴ are absent and R¹²C—CR¹³R¹⁴ is taken to be C═CR¹³; R¹⁴ and R¹⁶ are absent and R¹³R¹⁴C—CR¹⁵R¹⁶ is taken to be R¹³C═CR¹⁵; R¹⁶ and R¹⁸ are absent and R¹⁵R¹⁶C—CR¹⁷R¹⁸ is taken to be R¹⁵C═CR¹⁷; R¹⁸ and R²⁰ are absent and R¹⁷R¹⁸C—CR¹⁹R²⁰ is taken to be R¹⁷C═CR¹⁹; R²⁰ and R²² are absent and R¹⁹R²⁰C—CR²¹R²² is taken to be R¹⁹C═CR²¹; or R²² and R¹² are absent and R²¹R²²C—CR¹² is taken to be R²¹C═C;
when Y is cyclohexadiene, either: R¹², R¹⁴, R¹⁶ and R¹⁸ are absent and R¹²C—CR¹³R¹⁴ and R¹⁵R¹⁶C—CR¹⁷R¹⁸ are taken to be C═CR¹³ and R¹⁵C═CR¹⁷; R¹², R¹⁴, R¹⁸ and R²⁰ are absent and R¹²C—CR¹³R¹⁴ and R¹⁷R¹⁸C—CR¹⁹R²⁰ are taken to be C═CR¹³ and R¹⁷C═CR¹⁹; R¹², R¹⁴, R²⁰ and R²² are absent and R¹²C—CR¹³R¹⁴ and R¹⁹R²⁰C—CR²¹R²² are taken to be C═CR¹³ and R¹⁹C═CR²¹; R¹⁴, R¹⁶, R¹⁸ and R²⁰ are absent and R¹³R¹⁴C—CR¹⁵R¹⁶ and R¹⁷R¹⁸C—CR¹⁹R²⁰ are taken to be R¹³C═CR¹⁵ and R¹⁷C═CR¹⁹; R¹⁴, R¹⁶, R²⁰ and R²² are absent and R¹³R¹⁴C—CR¹⁵R¹⁶ and R¹⁹R²⁰C—CR²¹R²² are taken to be R¹³C═CR¹⁵ and R¹⁹C═CR²¹; R¹⁴, R¹⁶, R²² and R¹² are absent and R¹³R¹⁴C—CR¹⁵R¹⁶ and R²¹R²²C—CR¹² are taken to be R¹³C═CR¹⁵ and R²¹C═C; R¹⁶, R¹⁸, R²⁰ and R²² are absent and R¹⁵R¹⁶C—CR¹⁷R¹⁸ and R¹⁹R²⁰C—CR²¹R²² are taken to be R¹⁵C═CR¹⁷ and R¹⁹C═CR²¹; R¹⁶, R¹⁸, R²² and R¹² are absent and R¹⁵R¹⁶C—CR¹⁷R¹⁸ and R²¹R²²C—CR¹² are taken to be R¹⁵C═CR¹⁷ and R²¹C═C; or R¹⁸, R²⁰, R²² and R¹² are absent and R¹⁷R¹⁸C—CR¹⁹R²⁰ and R²¹R²²C—CR¹² are taken to be R¹⁷C═CR¹⁹ and R²¹C═C;
each of R¹ to R²² when present is otherwise independently selected from the group consisting of —H, —OH, halogen, —CN, —SH, —C₁-C₁₀ alkyl, —C₂-C₁₀ alkenyl, —C₂-C₁₀ alkynyl, —C(O)C₁-C₁₀ alkyl, —C(O)C₂-C₁₀ alkenyl, —C(O)C₂-C₁₀ alkynyl, —OC₁-C₁₀ alkyl, —OC₂-C₁₀ alkenyl, —OC₂-C₁₀ alkynyl, —OC(O)C₁-C₁₀ alkyl, —OC(O)C₂-C₁₀ alkenyl, —OC(O)C₂-C₁₀ alkynyl, —OC(O)NHC₁-C₁₀ alkyl, —OC(O)NHC₂-C₁₀ alkenyl, —OC(O)NHC₂-C₁₀ alkynyl, and —N(R²³)₂, or one or more of R¹³ and R¹⁴, R¹⁵ and R¹⁶, R¹⁷ and R¹⁸, R¹⁹ and R²⁰, and R²¹ and R²², taken together, are ═O, ═S, or ═NR²³;
each R²³ is independently selected from the group consisting of H and —C₁-C₁₀ alkyl; and
W is absent or selected from the group consisting of C₁-C₁₀ alkylene, C₂-C₁₀ alkenylene, C₂-C₁₀ alkynylene, C(O)C₁-C₁₀ alkylene, C(O)C₂-C₁₀ alkenylene, and C(O)C₂-C₁₀ alkynylene.

16. The method according to claim 15, wherein X is benzene.

17. The method according to claim 16, wherein each of R², R⁴, R⁶, R⁸ and R¹⁰ are independently selected from the group consisting of —H and —OH.

18. (canceled)

19. The method according to claim 15, wherein Y is cyclohexane, cyclohexene, or benzene.

20. The method according to claim 19, wherein each of R¹² to R²² when present, apart from the one that is —C₁-C₁₀ alkyl, are independently selected from the group consisting of —H, —OH, —C₁-C₁₀ alkyl, —C(O)C₁-C₁₀ alkyl, or one or more of R¹³ and R¹⁴, R¹⁵ and R¹⁶, R¹⁷ and R¹⁸, R¹⁹ and R²⁰, and R²¹ and R²², taken together, are ═O.

21. The method according to claim 20, wherein:
Y is benzene, R¹³ and R²¹ are independently selected from the group consisting of —C(O)C₁-C₁₀ alkyl and —C₁-C₁₀ alkyl, R¹⁵ and R¹⁹ are —OH, and R₁₇ is —C₁-C₁₀ alkyl;
Y is cyclohexane, R¹³, R¹⁴, R¹⁷, R¹⁸, R²¹ and R²² are independently selected from the group consisting of —H and —C₁-C₁₀ alkyl, at least one of which being —C₁-C₁₀ alkyl, and R¹², R¹⁵, R¹⁶, R¹⁹ and R²⁰ are independently selected from the group consisting of —H and —OH;
Y is cyclohexene, R¹⁴ and R¹⁶ are absent and R¹³R¹⁴C—CR¹⁵R¹⁶ is taken to be R¹³C═CR¹⁵, or R²⁰ and R²² are absent and R¹⁹R²⁰C—CR²¹R²² is taken to be R¹⁹C═CR²¹, R¹⁷ and R¹⁸ taken together are ═O, R¹² is selected from the group consisting of —H and —OH, at least one of R¹³, R¹⁴, R²¹ and R²² is —C₁-C₁₀ alkyl, and the remainder of R¹² to R²² are —H; or
Y is cyclohexene, R¹⁶ and R¹⁸ are absent and R¹⁵R¹⁶C—CR¹⁷R¹⁸ is taken to be R¹⁵C═CR¹⁷, R¹⁷ is —C₁-C₁₀ alkyl, and the remainder of R¹² to R²² are —H.

22. (canceled)

23. (canceled)

24. (canceled)

25. The method according to claim 15, wherein W is absent or selected from the group consisting of C₁-C₁₀ alkylene and C₂-C₁₀ alkenylene.

26. The method according to claim 15, wherein Z represents α- or β-, D- or L-, glucopyranose, mannopyranose or galactopyranose.

27. (canceled)

28. The method according to claim 15, wherein the compound is selected from the group consisting of:

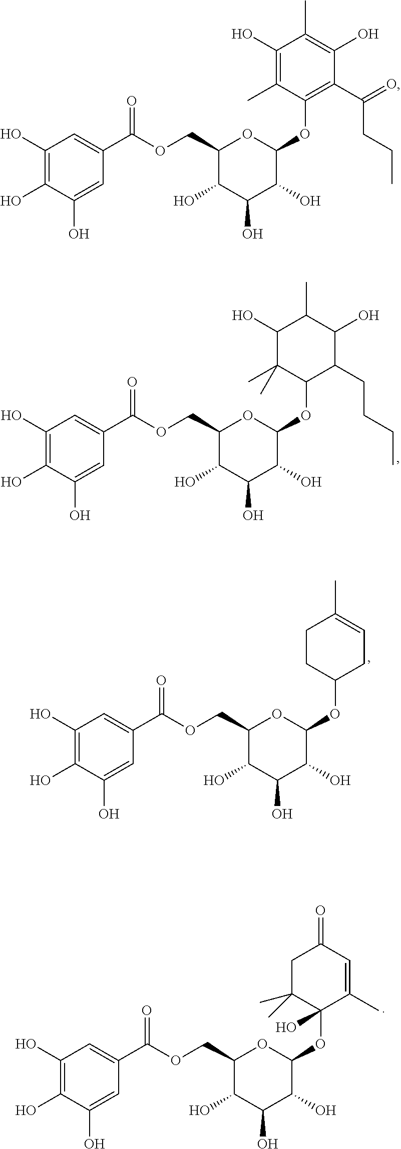 [SMILES: CCCC(=O)c1c(O)c(C)c(O)c(C)c1O[C@@H]1O[C@H](COC(=O)c2cc(O)c(O)c(O)c2)[C@@H](O)[C@H](O)[C@H]1O ; CCCCC1C(O)C(C)C(O)C(C)(C)C1O[C@@H]1O[C@H](COC(=O)c2cc(O)c(O)c(O)c2)[C@@H](O)[C@H](O)[C@H]1O ; CC1=CCC(O[C@@H]2O[C@H](COC(=O)c3cc(O)c(O)c(O)c3)[C@@H](O)[C@H](O)[C@H]2O)CC1 ; CC1=CC(=O)CC(C)(C)[C@@]1(O)O[C@@H]1O[C@H](COC(=O)c2cc(O)c(O)c(O)c2)[C@@H](O)[C@H](O)[C@H]1O]

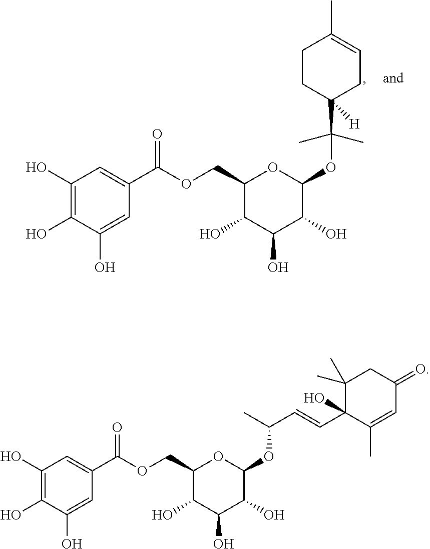

29. The method according to claim 15, wherein the inflammatory disease or disorder is associated with a cytokine, preferably an interleukin or an interferon.

30. The method according to claim 29, wherein the interleukin or an interferon is selected from one or more of the group consisting of IFN-γ, IL-17A and IL-8.

31. A pharmaceutical composition comprising a compound of Formula (II) or pharmaceutically acceptable salt as defined in claim 15.

32. (canceled)

33. (canceled)

34. (canceled)

35. (canceled)
